(19) United States
(12) Patent Application Publication
(10) Pub. No.: US 2026/0272422 A1
(43) Pub. Date: Sep. 17, 2026

(54) AUTOMATED DIAGNOSIS OF LUNG CONDITIONS USING IMAGES FROM AN ULTRASOUND SCANNER

(71) Applicant: Exo Imaging, Inc., Santa Clara, CA (US)

(72) Inventors: Amirhosein FOROUZANDEHMOGHADAM, Vancouver (CA); Seyed Ehsan SEYED BOLOURI, Burnaby (CA); Roberto Ivan VEGA ROMERO, Edmonton (CA); Mahdiar NEKOUI, Edmonton (CA)

(21) Appl. No.: 19/470,006

(22) PCT Filed: Mar. 20, 2024
(86) PCT No.: PCT/US2024/020776

§ 371 Date: Sep. 26, 2025

Related U.S. Application Data
(60) Provisional application No. 63/493,473, filed on Mar. 31, 2023.

(51) Int. Cl. A61B 8/00; A61B 8/08; G06T 7/12

(52) U.S. Cl. CPC A61B 8/5207; A61B 8/085; A61B 8/4427; A61B 8/4461; G06T 7/12; G06T 2207/10132; G06T 2207/30061

(57) ABSTRACT

A computing device inputs a plurality of ultrasound images of a lung into a segmentation model. For a respective image of the plurality of ultrasound images of the lung, the computing device obtains, from a first output of the segmentation model, a first segmented region of the respective image corresponding to a candidate A-line. The computing device compares the first segmented region in the respective image corresponding to the candidate A-line to other segmented regions from other images in the plurality of ultrasound images to determine a second segmented region of the respective image corresponding to an actual A-line. The computing device causes display of the second segmented region of the respective image.

### TECHNICAL FIELD

The disclosed implementations relate generally to systems, methods, and devices for utilizing an ultrasound probe.

### BACKGROUND

Ultrasound imaging is an imaging method that uses sound waves to produce images of structures or features within a region probed by the sound waves. In biological or medical applications, ultrasound images can be captured in real-time to show movement of internal organs as well as blood flowing through the blood vessels. The images can provide valuable information for diagnosing and directing treatment for a variety of diseases and conditions.

For lung ultrasounds, a variety of features can be diagnostically important. Identifying these features accurately and quickly can be challenging for clinicians.

In order to increase the ease and reliability of diagnoses, there is a need for improved methods of automatically detecting these and other features of lung ultrasound images.

### SUMMARY

Medical ultrasound is an imaging modality that is based on the reflection of propagating sound waves at the interface between different tissues. Advantages of ultrasound imaging with respect to other imaging modalities may include one or more of: (1) its non-invasive nature, (2) its reduced costs, (3) its portability, and (4) its ability to provide a good temporal resolution, for example on the order of millisecond or better. Point-of-care ultrasound (POCUS) may be used at bedside by healthcare providers as a real-time tool for answering clinical questions (e.g., whether a patient has developmental hip dysplasia). A trained clinician may perform both the task of acquiring and the task of interpreting ultrasound images, without the need for a radiologist to analyze ultrasound images acquired by a highly trained technician. Depending on specifics of the ultrasound examination, there may still be highly specialized training to learn different protocols for acquiring medically relevant images that are high quality images.

In some embodiments, after a patient is positioned in an appropriate way, a clinician positions the ultrasound probe on the body of the patient and manually starts looking for an appropriate (e.g., an optimal) image that allows the clinician to make an accurate diagnosis. Acquiring the proper image may be a time-consuming activity that is performed by trial-and-error, and it may require extensive knowledge of human anatomy. For the diagnosis of some conditions, the clinician may need to perform manual measurements on the acquired images. Further, fatigue caused by repetitive tasks in radiology may lead to an increase in the number of diagnostic errors and decreased diagnosis accuracy. There is therefore a need to develop a methodology that allows clinicians to automatically, quickly and reliably, identify and characterize anatomical structures of interest.

Computer aided diagnosis (CAD) systems may help clinicians acquire higher quality images, and automatically analyze and measure relevant characteristics in ultrasound images. The methods, systems, and devices described herein may have one or more advantages, including (1) the ability to incorporate medical-expert domain knowledge in the machine learning algorithms for an efficient learning with a limited number of instances, (2) the ability to interpret outputs of the machine learning algorithms, (3) the ability to provide relevant clinical information to a clinician and provide guidance to clinicians to help the clinician acquire better ultrasound images in less time.

In some embodiments, the methods, systems, and devices described herein provide a device agnostic interpretation of lung ultrasound images. Lung ultrasound imaging data can include features (actual features) that are physically present in the lungs, such as pleural effusion and consolidation, as well as artifacts that are not physically present in the lungs, such as A-lines and B-lines. For example, lung ultrasounds typically include a bright pleural line indicative of the pleura, which consist of thin layers of tissue that protect and cushion the lungs. A-lines are repetitive horizontal echoic lines that arise from the pleural line at regular intervals (e.g., a skin-pleural line distance) that indicate subpleural air, which reflects the ultrasound beam. B-lines (also known as ultrasound lung comets) are due to vertical reverberation artefacts that originate from the pleural line, and generally extend to the depth of the image without substantially decreasing in intensity. B-lines also move synchronously with lung sliding. Both the physical features and the artifacts can provide an indication of the health of the lungs.

In some embodiments, the methods, systems, and devices described herein utilize workflows, machine learning algorithms, and trained machine learning models (e.g., segmentation models) to accurately and efficiently detect the presence of A-lines and B-lines in the lung ultrasound data, thereby enabling the detection of normal and abnormal lung conditions. In some embodiments, analogous techniques can be developed to utilize other physical features and/or artifacts that are later determined to be relevant to the diagnosis of various conditions of the lung from lung ultrasound data.

In some embodiments, the methods, systems, and devices described herein provide an identification of segmented regions corresponding to an ultrasound image and/or a classification of the image quality (e.g., good quality, moderate quality, or bad quality) in real time, during an ultrasound scan. Accordingly, in some embodiments, the disclosed methods, systems, and devices advantageously improve user experience by providing real time feedback to a user and assisting the user to obtain high-quality images that contain the features of interest.

The systems, methods, and devices of this disclosure have several innovative aspects, the desirable attributes disclosed herein may be derived from one or more of the innovative aspects individually or as a combination, in accordance with some embodiments.

In accordance with some embodiments, a method of detecting presence of A-lines in ultrasound images is performed at a computer device that includes one or more processors and memory. The method includes inputting a plurality of ultrasound images of a lung into a segmentation model. The method includes, for a respective image of the plurality of ultrasound images of the lung, obtaining, from a first output of the segmentation model, a first segmented region of the respective image corresponding to a candidate A-line. The method includes comparing the first segmented region in the respective image corresponding to the candidate A-line to other segmented regions from other images in the plurality of ultrasound images to determine a second segmented region of the respective image corresponding to an actual A-line. The method includes causing display of the second segmented region of the respective image.

In some embodiments, the segmentation model is a multi-headed model. The multi-headed model includes a first head of the multi-headed model configured to output the first output; and a second head of the multi-headed model configured to output a second output comprising a quality metric associated with the ultrasound image of the lung.

In some embodiments, the first output comprises, for each respective portion of a plurality of portions of the image, a respective segmentation value of a plurality of segmentation values.

In some embodiments, the method further includes obtaining, from the segmentation model, a third segmented region of the respective image corresponding to a pleura of the lung; using the third segmented region to generate a mask; and using the mask to detect the presence or absence of B-lines in the respective image.

In some embodiments, causing display of the second segmented region of the respective image includes causing the third segmented region to be displayed concurrently with the second segmented region in the respective image.

In some embodiments, the method includes obtaining, from the segmentation model, a fourth segmented region of the respective image corresponding to one or more ribs adjacent to the pleura of the lung.

In some embodiments, causing display of the second segmented region of the respective image includes causing the third segmented region and the fourth segmented region to be displayed concurrently with the second segmented region in the respective image.

In some embodiments, the respective image comprises a plurality of pixels. The segmentation model is configured to output, for a respective pixel of the plurality of pixels, a respective segmentation value corresponding to the respective pixel.

In some embodiments, the respective segmentation value is one of a plurality of segmentation values that the segmentation model is configured to output. The plurality of segmentation values includes a first segmentation value corresponding to a background, a second segmentation value corresponding to a pleura, and a third segmentation value corresponding to the A-line.

In some embodiments, the plurality of segmentation values includes a fourth segmentation value corresponding to one or more ribs adjacent to the pleura.

In some embodiments, obtaining the first segmented region of the respective image corresponding to the candidate A-line includes identifying, from the plurality of pixels, a first group of pixels that are contiguously located on the respective image. Each pixel of the first group of pixels has the same segmentation value corresponding to the A-line.

In some embodiments, the respective image and the other images form a sequence of images in the plurality of ultrasound images.

In some embodiments, the method includes, prior to inputting the plurality of ultrasound images into the segmentation model, pre-processing the plurality of ultrasound images. The pre-processing includes at least one of: normalizing respective intensities of the plurality of ultrasound images and adjusting respective image sizes of the plurality of ultrasound images.

In some embodiments, the method includes obtaining a first region corresponding to the respective image, the first region having a plurality of columns of pixels. The method includes comparing respective intensity values from a respective column of pixels, of the plurality of columns of pixels, with other intensity values from other columns of pixels, of the plurality of columns of pixels in the first region; and determining one or more candidate B-lines in the first region in accordance with the comparing.

In some embodiments, the method includes applying a mask to the one or more candidate B-lines in the first region to obtain one or more actual B-lines in the first region.

In some embodiments, the method includes causing the one or more actual B-lines to be displayed in the respective image.

In some embodiments, the respective image has a first coordinate system. The first region is determined relative to a second coordinate system, distinct from the first coordinate system. Causing display of the one or more actual B-lines in the respective image comprises transforming the one or more actual B-lines from the second coordinate system to the first coordinate system.

In some embodiments, the first region is obtained by transforming a first portion of the respective image, the first region having a rectilinear shape and the first portion having a conical shape.

In accordance with some embodiments, a method of determining scan quality of lung ultrasound images is performed at a computer device that includes one or more processors and memory. The method includes, during a first scan performed by an ultrasound probe, receiving a plurality of ultrasound images of a lung acquired via the ultrasound probe. The method includes inputting a respective ultrasound image of the plurality of ultrasound images into a multi-headed model. The method includes receiving, from a first head of the multi-headed model, a first output comprising one or more segmented regions of the respective image. The method includes receiving, from a second head of the multi-headed model, a second output comprising a quality metric associated with the respective image. The method includes causing concurrent display of the quality metric and the one or more segmented regions.

In some embodiments, a method of determining scan quality of lung ultrasound images acquired via an ultrasound probe comprises, during a first scan performed by the ultrasound probe, receiving a plurality of ultrasound images of a lung acquired via the ultrasound probe. The method includes, while receiving the plurality of ultrasound images, for each ultrasound image of the plurality of ultrasound images: inputting a respective ultrasound image of the plurality of ultrasound images into a multi-headed model. The method includes receiving, from a first head of the multi-headed model, a first output comprising one or more segmented regions of the respective image. The method includes receiving, from a second head of the multi-headed model, a second output comprising a quality score associated with the respective image, the quality score indicative of a quality of the respective image. The method includes causing concurrent display of the quality score and the one or more segmented regions.

In some embodiments, the method includes, in accordance with a determination the plurality of ultrasound images includes a threshold number of ultrasound images that each satisfies a threshold quality score: automatically recording a stack of first ultrasound images of the lung acquired via the ultrasound probe.

In some embodiments, the method includes, while automatically recording of the stack of first ultrasound images, for each first ultrasound image of the stack of first ultrasound images: in accordance with a determination that the respective first ultrasound image satisfies the threshold quality score: in accordance with a determination that a number of the first ultrasound images in the stack is less than a threshold number, continuing the automatic recording of the stack; and in accordance with a determination that the number of the first ultrasound images in the stack satisfies the threshold number, ceasing the automatic recording of the stack.

In some embodiments, the method includes, while automatically recording of the stack of first ultrasound images, for each first ultrasound image of the stack of first ultrasound images: in accordance with a determination that the respective first ultrasound image does not satisfy the threshold quality score: (i) discarding the respective image; and (ii) continuing to record the stack of first ultrasound images.

In some embodiments, the method includes, while automatically recording the stack of first ultrasound images of the lung, obtaining diagnostic information of the lung based on the stack of ultrasound images.

In some embodiments, the method includes, in accordance with a determination that the number of the first ultrasound images in the stack satisfies a threshold number: (i) ceasing the automatic recording of the stack; and (ii) causing display of a diagnosis of the lung.

In accordance with some embodiments, a method of detecting presence of B-lines in ultrasound images includes inputting a plurality of ultrasound images of a lung into a segmentation model. The method includes, for a respective image of the plurality of ultrasound images of the lung, obtaining, from the segmentation model, a set of vertices. The method includes, in accordance with a determination that each vertex of the set of vertices corresponds to a respective corner of a B-line in the respective ultrasound image, (i) generating a segmented region of the respective image corresponding to the B-line; and (ii) causing display of the segmented region with the respective image.

In some embodiments, the set of vertices comprises a plurality of vertices. The segmentation model comprises a plurality of segmentation sub-models. Each segmentation sub-model of the plurality of segmentation sub-models is configured to determine one respective vertex of the plurality of vertices.

In accordance with some embodiments, a computer device (e.g., computer system) comprises one or more processors and memory. The memory stores memory storing instructions that, when executed by the one or more processors, cause the computer system to perform any of the methods disclosed herein.

In accordance with some embodiments of the present disclosure, a non-transitory computer readable storage medium stores computer-executable instructions. The computer-executable instructions, when executed by one or more processors of a computer device (e.g., computer system), cause the computer device to perform any of the methods disclosed herein.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates an exemplary operating environment, in accordance with some embodiments.

FIG. 2 illustrates a block diagram of an ultrasound device in accordance with some embodiments.

FIG. 3 illustrates a block diagram of a computing device in accordance with some embodiments.

FIG. 4 illustrates a workflow for detecting A-lines from ultrasound images of a lung, in accordance with some embodiments.

FIG. 5 illustrates a temporal sequence of ultrasound images of a lung, in accordance with some embodiments.

FIG. 6 shows an ultrasound image of a lung that includes multiple A-lines.

FIG. 7 illustrates a workflow for detecting B-lines in ultrasound images of a lung, in accordance with some embodiments.

FIGS. 8A and 8B illustrate ultrasound images and corresponding pleura masks, in accordance with some embodiments.

FIG. 9 illustrates respective fields of view for different types of ultrasound probes, in accordance with some embodiments.

FIGS. 10A and 10B illustrate an ultrasound image that is acquired using a curvilinear probe, in accordance with some embodiments.

FIGS. 11A and 11I illustrate an exemplary process for converting a field of view from a cone/wedge shape to a rectilinear shape, in accordance with some embodiments.

FIG. 12 illustrates a process for obtaining actual B-lines, performed by a pleura line filter, in accordance with some embodiments.

FIG. 13 illustrates a workflow for acquiring ultrasound scan data, in accordance with some embodiments.

FIG. 14 illustrates an exemplary process for detecting B-lines from lung ultrasound images, in accordance with some embodiments.

FIGS. 15A to 15G illustrate an example user interface for displaying ultrasound scan data and/or other feedback, in accordance with some embodiments.

FIGS. 16A to 16D illustrate a flowchart diagram for a method of detecting the presence of A-lines in ultrasound images, in accordance with some embodiments.

FIG. 17 illustrates a flowchart diagram for a method of determining scan quality of lung ultrasound images, in accordance with some embodiments.

FIGS. 18A and 18B illustrate a flowchart diagram for a method of determining scan quality of lung ultrasound images acquired via an ultrasound probe, in accordance with some embodiments.

FIG. 19 illustrates a flowchart diagram for a method of detecting presence of B-lines in ultrasound images, in accordance with some embodiments.

Reference will now be made to implementations, examples of which are illustrated in the accompanying drawings. In the following description, numerous specific details are set forth in order to provide a thorough understanding of the present invention. However, it will be apparent to one of ordinary skill in the art that the present invention may be practiced without requiring some of these specific details.

### DESCRIPTION OF IMPLEMENTATIONS

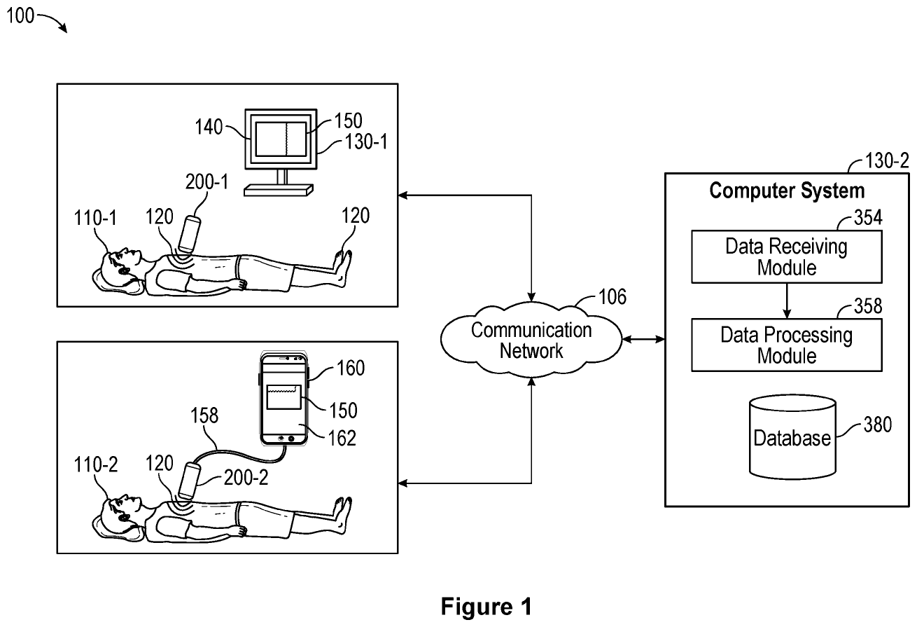

FIG. 1 illustrates an exemplary operating environment 100, in accordance with some embodiments.

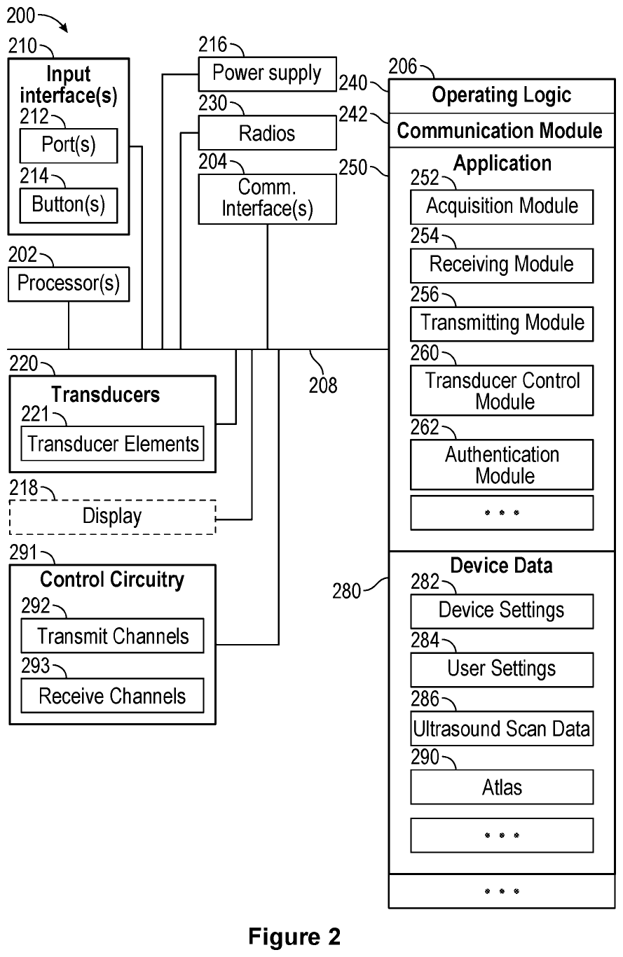

In some embodiments, the operating environment 100 includes one or more ultrasound devices 200 (e.g., ultrasound device 200-1 and ultrasound device 200-2). According to some embodiments of the present disclosure, the ultrasound device 200 is also referred to as an ultrasound imaging device or an ultrasound imaging system. In some embodiments, the ultrasound device 200 is a portable handheld device. In some embodiments, the ultrasound device 200 includes a probe portion that includes transducers (e.g., transducers 220, FIG. 2). In some embodiments, the transducers are arranged in an array. In some embodiments, the ultrasound device 200 includes an integrated control unit and user interface. In some embodiments, the ultrasound device 200 includes a probe that communicates with a control unit and user interface that is external to the housing of the probe itself. During operation, the ultrasound device 200 (e.g., via the transducers) produces sound waves 120 that are transmitted toward an organ, such as a heart or a lung, of a respective patient 110 (e.g., patient 110-1 or patient 110-2). The internal organ, or other object(s) to be imaged, may reflect a portion of the sound waves toward the probe portion of the ultrasound device 200, which are received by the transducers 220.

In some embodiments, the ultrasound device 200 uses the received signals to generate an ultrasound image that is displayed on a display (e.g., display 218, FIG. 2) of the ultrasound device. In some embodiments, the ultrasound device 200 uses the received signals to generate an ultrasound image that is displayed on a display 162 of an electronic device 160 (e.g., a computing device) that is communicatively connected (e.g., wirelessly or via a wired connection, such as cable 158) to the ultrasound device.

In some embodiments, the ultrasound device 200 is communicatively coupled through communication network(s) 106 to a computer system 130 (e.g., a computer device). For example, the ultrasound device 200 transmits the received signals to computer system 130, which uses the received signals to create an image 150 that is also known as a sonogram. In some embodiments, the computer system 130 includes a display 140 (e.g., a display device) for displaying ultrasound images, and other input and output devices (e.g., keyboard, touch screen, joystick, touchpad, and/or speakers).

In some embodiments, the computer system 130 and the ultrasound device 200 are co-located at the same location (e.g., in the same room where the ultrasound device 200 is deployed, such as ultrasound device 200-1 and computer system 130-1 in FIG. 1). In some embodiments, the ultrasound device 200 and the computer system 130 are located at different locations (e.g., such as computer system 130-2 in FIG. 1).

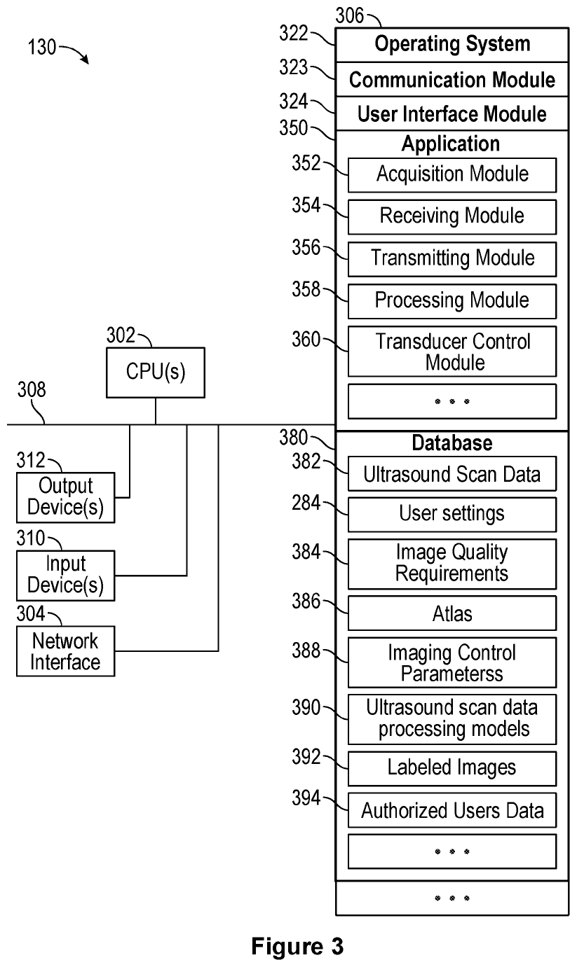

In some embodiments, the computer system 130 includes a data receiving module 354 that is configured to receive data from one or more ultrasound devices 200, such as ultrasound medical imaging data and/or ultrasound data for user authentication. The computer system 130 includes a data processing module 358 that is configured to process the received data. In some embodiments, the computer system 130 includes a database 380. Further details of the data receiving module 354, data processing module 358, and database 380 are discussed with respect to FIG. 3.

FIG. 2 illustrates a block diagram of an exemplary ultrasound device 200 in accordance with some embodiments.

The ultrasound device 200 may be used to perform different types of imaging. For example, the ultrasound device 200 may be used to perform one-dimensional imaging, also known as A-Scan, two-dimensional imaging, also known as B scan, three-dimensional imaging, also known as C scan, and Doppler imaging. The ultrasound device 200 may be switched to different imaging modes, including without limitation linear mode and sector mode, and electronically configured under program control.

In some embodiments, the ultrasound device 200 includes one or more processors 202, one or more communication interfaces 204 (e.g., network interface(s)), memory 206, and one or more communication buses 208 for interconnecting these components (sometimes called a chipset).

In some embodiments, the ultrasound device 200 includes one or more input interfaces 210 that facilitate user input. For example, in some embodiments, the input interfaces 210 include port(s) 212 and button(s) 214. In some embodiments, the port(s) can be used for receiving a cable (e.g., cable 158) for powering or charging the ultrasound device 200, or for facilitating communication between the ultrasound device and other devices (e.g., computer system 130, electronic device 160, display device 140, printing device, and/or other input output devices and accessories).

In some embodiments, the ultrasound device 200 includes a power supply 216. For example, in some embodiments, the ultrasound device 200 is battery-powered. In some embodiments, the ultrasound device is powered by a continuous AC power supply.

In some embodiments, the ultrasound device 200 includes a display 218 for displaying ultrasound imaging data and/or other notifications generated by the ultrasound device 200.

In some embodiments, the ultrasound device 200 includes a probe portion that includes transducers 220, which may also be referred to as transceivers or imagers. Examples of transducers 220 include, without limitation, piezoelectric micromachined ultrasonic transducers (PMUT) and capacitive micromachined ultrasonic transducers (CMUT). In some embodiments, the transducers 220 are based on photo-acoustic or ultrasonic effects. For ultrasound imaging, the transducers 220 generate and transmit ultrasonic waves towards a target (e.g., a target organ, blood vessels, etc.) to be imaged. The transducers 220 receive reflected sound waves (e.g., echoes) that bounce off body tissues. The reflected waves are then converted to electrical signals and/or ultrasound images. In some embodiments, the probe portion of the ultrasound device 200 is separately housed from the computing and control portion of the ultrasound device. In some embodiments, the probe portion of the ultrasound device 200 is integrated in the same housing as the computing and control portion of the ultrasound device 200. In some embodiments, part of the computing and control portion of the ultrasound device is integrated in the same housing as the probe portion, and part of the computing and control portion of the ultrasound device is implemented in a separate housing that is coupled communicatively with the part integrated with the probe portion of the ultrasound device. In some embodiments, the probe portion of the ultrasound device has a respective transducer array that is tailored to a respective scanner type (e.g., linear, convex, endocavitary, phased array, transesophageal, 3D, and/or 4D). In the present disclosure, “ultrasound probe” may refer to the probe portion of an ultrasound device, or an ultrasound device that includes a probe portion.

In some embodiments as used herein, a transducer 220 is also known as a “pixel.” In some embodiments, multiple pixels/transducers (e.g., 4, 8, 16, or 64) are grouped together to form a transducer element 221. For example, in some embodiments, the ultrasound device 200 is configured to control transmit (Tx) firings (the transmissions of ultrasonic waveforms from a transducer pixel or a transducer element) by sequentially activating one transducer 220 at a time. In some embodiments, the ultrasound device 200 is configured to control Tx firings by sequentially activating two or more transducers (e.g., a transducer element 221) at a time.

In some embodiments, the ultrasound device 200 includes a control circuitry 291 that is configured to control transducers 220 and/or transducer elements 221 to send ultrasound waves toward the target object being imaged while other transducers 220 and/or transducer elements 221, at the same time, receive the ultrasound waves/ultrasonic energy reflected from the target object, and generate electrical charges based on the same in response to the received ultrasound waves/ultrasonic energy. To generate the pressure waves, the ultrasound device 200 may include a number of transmit (Tx) channels 292 and a number of receive (Rx) channels 293. The transmit channels 292 may include a number of components that drive the transducers 220 (e.g., transducer elements 221) with a voltage pulse at a frequency that they are responsive to. This causes an ultrasonic waveform to be emitted from the transducers 220 towards an object to be imaged.

In some embodiments, the ultrasound device 200 includes radios 230. The radios 230 enable one or more communication networks, and allow the ultrasound device 200 to communicate with other devices, such as the computer system 130 in FIGS. 1 and 3, the display device 140 in FIG. 1, and/or the electronic device 160 in FIG. 1. In some implementations, the radios 230 are capable of data communications using any of a variety of custom or standard wireless protocols (e.g., IEEE 802.15.4, Wi-Fi, ZigBee, 6LoWPAN, Thread, Z-Wave, Bluetooth Smart, ISA100.5A, WirelessHART, MiWi, Ultrawide Band (UWB), software defined radio (SDR) etc.) custom or standard wired protocols (e.g., Ethernet, HomePlug, etc.), and/or any other suitable communication protocol, including communication protocols not yet developed as of the filing date of this document.

The memory 206 includes high-speed random access memory, such as DRAM, SRAM, DDR RAM, or other random access solid state memory devices; and, optionally, includes non-volatile memory, such as one or more magnetic disk storage devices, one or more optical disk storage devices, one or more flash memory devices, or one or more other non-volatile solid state storage devices. The memory 206, optionally, includes one or more storage devices remotely located from one or more processor(s) 202. The memory 206, or alternatively the non-volatile memory within the memory 206, includes a non-transitory computer-readable storage medium. In some implementations, the memory 206, or the non-transitory computer-readable storage medium of the memory 206, stores the following programs, modules, and data structures, or a subset or superset thereof:
operating logic 240 including procedures for handling various basic system services and for performing hardware dependent tasks;
a communication module 242 (e.g., a radio communication module) for connecting to and communicating with other network devices (e.g., a local network, such as a router that provides Internet connectivity, networked storage devices, network routing devices, server systems, computer device 130, electronic device 160, and/or other connected devices etc.) coupled to one or more communication networks via the communication interface(s) 204 (e.g., wired or wireless);
application 250 for acquiring ultrasound data (e.g., imaging data) of a patient, and/or for controlling one or more components of the ultrasound device 200 and/or other connected devices (e.g., in accordance with a determination that the ultrasound data meets, or does not meet, certain conditions). In some embodiments, the application 250 includes:
   an acquisition module 252 for acquiring ultrasound data (e.g., via control circuitry 291 and transmit channels 292). In some embodiments, the ultrasound data includes imaging data. In some embodiments, the acquisition module 252 activates the transducers 220 (e.g., less than all of the transducers 220, different subset(s) of the transducers 220, all the transducers 220, transducer elements 221, etc.) according to whether the ultrasound data meets one or more conditions associated with one or more quality requirements;
   a receiving module 254 for receiving ultrasound data (e.g., via control circuitry 291 and receive channels 293);
   a transmitting module 256 for transmitting ultrasound data to other device(s) (e.g., a server system, computer device 130, display device 140, electronic device 160, and/or other connected devices etc.);
   a transducer control module 260 for adjusting imaging control parameters (e.g., between an authentication scan and a diagnostic scan, or between diagnostic scans of different anatomies);
   an authentication module 262 for authenticating a user of the ultrasound device 200 (e.g., by transmitting the device to a computer system 130 for authentication). In some embodiments, the authentication module 262 is used to provision the ultrasound device 200 (e.g., during initial setup) and/or to associate additional users with the ultrasound device 200;
device data 280 for the ultrasound device 200, including but not limited to:
   device settings 282 for the ultrasound device 200, such as default options and preferred user settings. In some embodiments, the device settings 282 include imaging control parameters. For example, in some embodiments, the imaging control parameters include one or more of: a number of transducers that are activated, a power consumption threshold of the probe, an imaging frame rate, a scan speed, a depth of penetration, and other scan parameters that control the power consumption, heat generation rate, and/or processing load of the probe;
   user settings 284, such as a preferred gain, depth, zoom, and/or focus settings. In some embodiments, user settings are loaded automatically (e.g., without user intervention) after the user authenticates using an ultrasound scan as described herein;
   ultrasound scan data 286 (e.g., imaging data) that are acquired (e.g., detected, measured) by the ultrasound device 200 (e.g., via transducers 220);
   an atlas 290. In some embodiments, the atlas 290 includes anatomical structures of interest. In some embodiments, the atlas 290 includes three-dimensional representations of the anatomical structure of interest (e.g., hip, heart, lung, and/or other anatomical structures);

Each of the above identified executable modules, applications, or sets of procedures may be stored in one or more of the previously mentioned memory devices, and corresponds to a set of instructions for performing a function described above. The above identified modules or programs (i.e., sets of instructions) need not be implemented as separate software programs, procedures, or modules, and thus various subsets of these modules may be combined or otherwise re-arranged in various implementations. In some implementations, the memory 206 stores a subset of the modules and data structures identified above. Furthermore, the memory 206 may store additional modules or data structures not described above. In some embodiments, a subset of the programs, modules, and/or data stored in the memory 206 are stored on and/or executed by a server system, and/or by an external device (e.g., computer system 130, display device 140, or electronic device 160).

FIG. 3 illustrates a block diagram of a computer system 130 in accordance with some embodiments.

In some embodiments, the computer system 130 is a server or control console that is in communication with the ultrasound device 200 (e.g., ultrasound probe). In some embodiments, the computer system 130 is integrated into the same housing as the ultrasound device 200. In some embodiments, the computer system 130 is a smartphone, tablet device or other portable computing devices. In some embodiments, the computer system 130 may be provided by a combination of components integrated into the same housing as the ultrasound device 200, and a smartphone, tablet device, or other portable computing devices.

The computer system 130 includes one or more processors 302 (e.g., processing units of CPU(s)), one or more network interfaces 304, memory 306, and one or more communication buses 308 for interconnecting these components (sometimes called a chipset), in accordance with some implementations.

In some embodiments, the computer system 130 includes one or more input devices 310 that facilitate user input, such as a keyboard, a mouse, a voice-command input unit or microphone, a touch screen display, a touch-sensitive input pad, a gesture capturing camera, or other input buttons or controls. In some embodiments, the computer system 130 uses a microphone and voice recognition or a camera and gesture recognition to supplement or replace the keyboard. In some embodiments, the computer system 130 includes one or more output devices 312 that enable presentation of user interfaces and display content, such as one or more speakers and/or one or more visual displays (e.g., display device 140).

The memory 306 includes high-speed random access memory, such as DRAM, SRAM, DDR RAM, or other random access solid state memory devices; and, optionally, includes non-volatile memory, such as one or more magnetic disk storage devices, one or more optical disk storage devices, one or more flash memory devices, or one or more other non-volatile solid state storage devices. The memory 306, optionally, includes one or more storage devices remotely located from the one or more processors 302. The memory 306, or alternatively the non-volatile memory within the memory 306, includes a non-transitory computer-readable storage medium. In some implementations, the memory 306, or the non-transitory computer-readable storage medium of the memory 306, stores the following programs, modules, and data structures, or a subset or superset thereof:
an operating system 322 including procedures for handling various basic system services and for performing hardware dependent tasks;
a communication module 323 (e.g., a radio communication module) for connecting to and communicating with other network devices (e.g., a local network, such as a router that provides Internet connectivity, networked storage devices, network routing devices, server systems, computer device 130, ultrasound device 200, and/or other connected devices etc.) coupled to one or more communication networks via the network interface 304 (e.g., wired or wireless);
a user interface module 324 for enabling presentation of information (e.g., a graphical user interface for presenting application(s), widgets, websites and web pages thereof, games, audio and/or video content, text, etc.) either at the computer system 130 or another device;
application 350 for acquiring ultrasound data (e.g., imaging data) from a patient. In some embodiments, the application 350 is used for receiving data (e.g., ultrasound data, imaging data, etc.) acquired via an ultrasound device 200. In some embodiments, the application 350 is used for controlling one or more components of an ultrasound device 200 (e.g., the probe portion, and/or the transducers) and/or other connected devices (e.g., in accordance with a determination that the data meets, or does not meet, certain conditions). In some embodiments, the application 350 includes:
   an acquisition module 352 for acquiring ultrasound data. In some embodiments, the ultrasound data includes imaging data acquired by an ultrasound probe;
   a receiving module 354 for receiving ultrasound scan data. In some embodiments, the ultrasound scan data includes imaging data acquired by an ultrasound probe;
   a transmitting module 356 for transmitting ultrasound data (e.g., imaging data) to other device(s) (e.g., a server system, computer device 130, display device 140, ultrasound device 200, and/or other connected devices etc.);
   a processing module 358 for processing ultrasound data and/or other data; and
   a transducer control module 360 for adjusting imaging control parameters (e.g., between an authentication scan and a diagnostic scan, or between diagnostic scans of different anatomies); and
a database 380, including:
   ultrasound scan data 382 (e.g., imaging data) that are acquired (e.g., detected, measured) by one or more ultrasound devices 200;
   user settings 284, such as a preferred gain, depth, zoom, and/or focus settings. In some embodiments, user settings are loaded automatically (e.g., without user intervention) after the user authenticates or logs onto a computing device that is communicatively connected with the ultrasound device 200;
   image quality requirements data 384. In some embodiments, the image quality requirements data 384 include clinical requirements for determining the quality of an ultrasound image;
   an atlas 386. In some embodiments, the atlas 386 includes anatomical structures of interest. In some embodiments, the atlas 386 includes three-dimensional representations of the anatomical structure of interest (e.g., hip, heart, or lung);
   imaging control parameters 388. For example, in some embodiments, the imaging control parameters include one or more of: a number of transducers that are activated, a power consumption threshold of the probe, an imaging frame rate, a scan speed, a depth of penetration, and other scan parameters that control the power consumption, heat generation rate, and/or processing load of the probe;
   ultrasound scan data processing models 390 for processing ultrasound data. For example, in some embodiments, the ultrasound scan data processing models 390 are trained neural network models that are trained to determine whether an ultrasound image meets quality requirements corresponding to a scan type, or trained to output an anatomic plane corresponding to an anatomical structure of an ultrasound image, or trained to predict, based on a sequence of ultrasound images and their quality scores, whether a subsequent frame to be acquired by an ultrasound probe will contain certain anatomical structures and/or landmarks of interest;
   labeled images 392 (e.g., a databank of images), including images for training the models that are used for processing new ultrasound data, and/or new images that have been or need to be processed. In some embodiments, the labeled images 392 are images of anatomical structures that have been labeled with their respective identifiers and relative positions; and
   authorized users data 394, including data of users that are authorized to use a respective ultrasound device. For example, in some embodiments, the authorized users data 394 are categorized according to respective identifiers corresponding to respective ultrasound devices. A respective identifier can be associated with one or more authorized subjects;

Each of the above identified elements may be stored in one or more of the memory devices described herein, and corresponds to a set of instructions for performing the functions described above. The above identified modules or programs need not be implemented as separate software programs, procedures, modules or data structures, and thus various subsets of these modules may be combined or otherwise re-arranged in various embodiments. In some embodiments, the memory 306, optionally, stores a subset of the modules and data structures identified above. Furthermore, the memory 306 optionally stores additional modules and data structures not described above. In some embodiments, a subset of the programs, modules, and/or data stored in the memory 306 are stored on and/or executed by the ultrasound device 200. In some embodiments, a subset of the programs, modules, and/or data stored in the memory 306 are stored on and/or executed by the electronic device 160.

### Automated Diagnosis of Lung Conditions

Lung ultrasound imaging data can include features (physical features) that are physically present in the lungs, such as the pleura (e.g., pleural line), pleural effusion and consolidation. Lung ultrasound data can also include artifacts that are not physically present in the lungs, such as A-lines and B-lines.

Both the physical features and the artifacts can provide an indication of the health of the lungs. For example, A-lines are repetitive horizontal echoic lines that arise from the pleura at regular intervals. A-lines occur when subpleural air in the lung reflects the ultrasound beam. The presence of A-lines in a lung ultrasound image is indicative of a healthy lung. B-lines are due to vertical reverberation artefacts that originate from the pleural line. The presence of B-lines in a lung ultrasound image indicates thickening of the pleural lining, e.g., due to infection or excess fluids, and are indicative of an unhealthy lung.

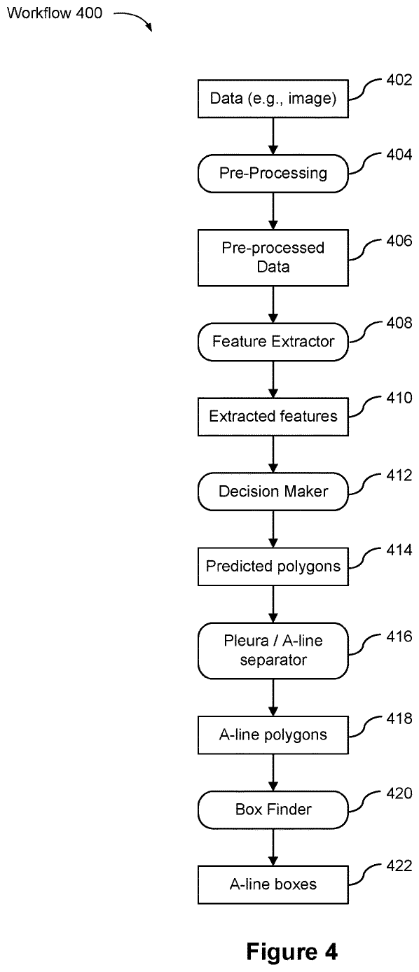

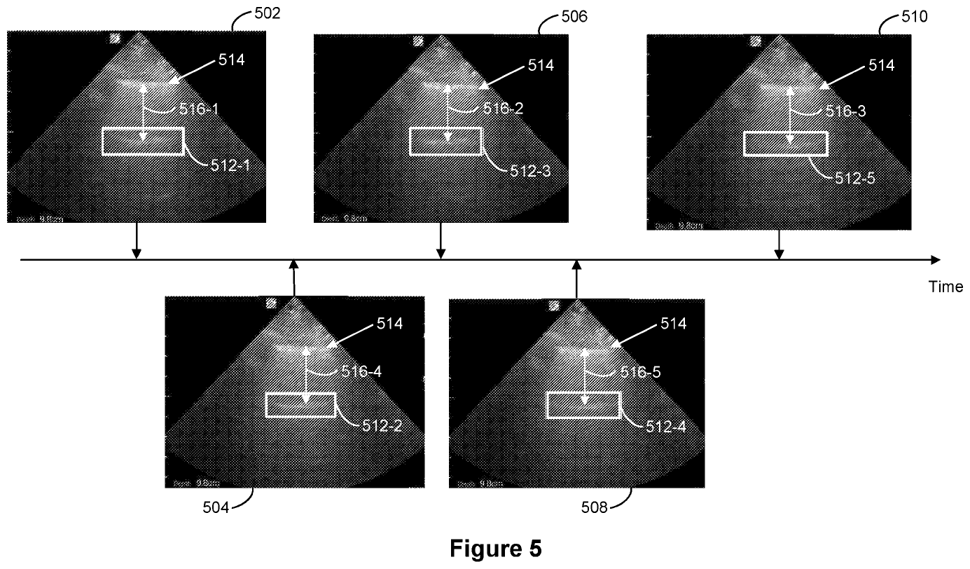

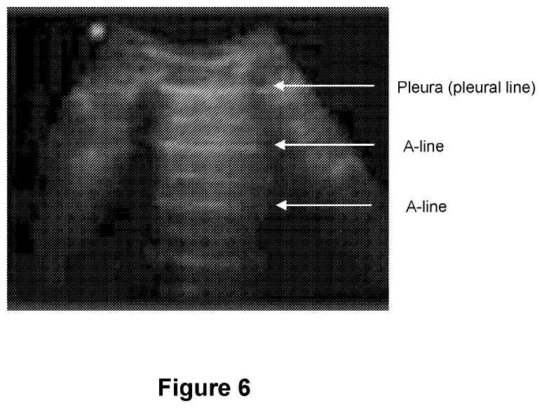

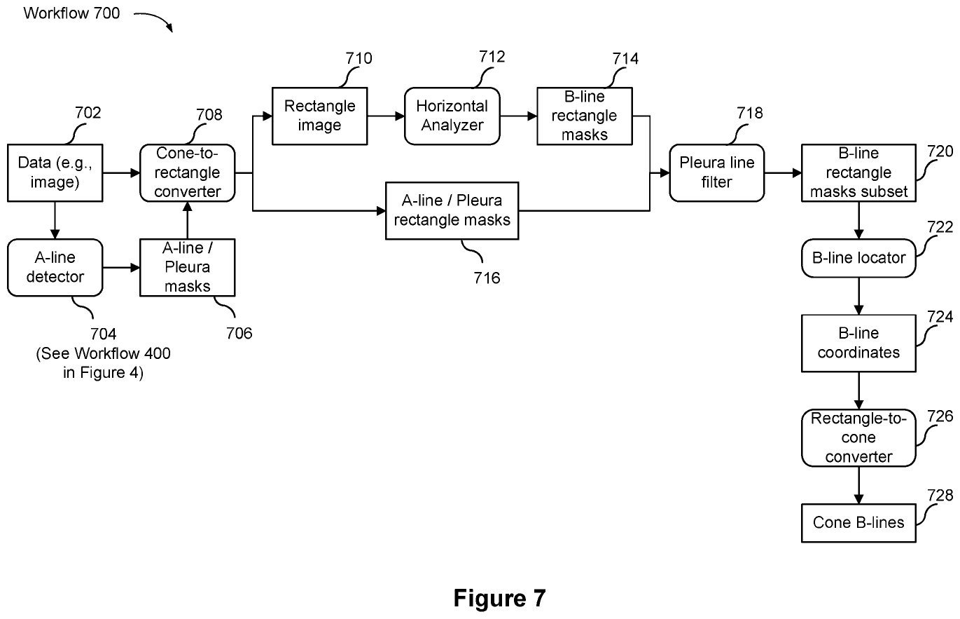

FIG. 4 illustrates a workflow 400 for detecting A-lines from ultrasound images of a lung, in accordance with some embodiments. In some embodiments, the workflow 400 is performed by one or more processors (e.g., CPU(s) 302) of a computing device that is communicatively connected with an ultrasound probe. For example, in some embodiments, the computing device is a server or control console (e.g., a server, a standalone computer, a workstation, a smart phone, a tablet device, a medical system) that is in communication with the ultrasound probe. In some embodiments, the computing device is a control unit integrated into the ultrasound probe. In some embodiments, the ultrasound probe is a handheld ultrasound probe or an ultrasound scanning system.

The workflow 400 includes obtaining (e.g., receiving) input data 402. In some embodiments, the workflow 400 is performed without user intervention in response to receiving the input data 402. In some embodiments, the input data comprises a series of ultrasound images (e.g., an image dataset) that includes a lung. Note that, throughout this disclosure, a series of images is sometimes referred to as a “sweep” when the images are obtained via single scan over (e.g., a single movement of the ultrasound probe of) an anatomy. In some embodiments, the input 402 is a sweep. In some embodiments, the computing device pre-processes the input data 402 via a pre-processing step 404 to obtain pre-processed data 406.

In some embodiments, the pre-processing includes adjusting a contrast and/or brightness of the ultrasound image(s) (e.g., to normalize respective intensities). In some embodiments, the pre-processing includes adjusting a respective image size of the ultrasound image(s). In some embodiments, the pre-processed data 406 comprises ultrasound images of the lung whose intensity values have been normalized and/or whose sizes have been adjusted (e.g., standardized).

In some embodiments, the workflow 400 includes applying a feature extractor 408 to extract features (extracted features 410) from the pre-processed data 406. In some embodiments, the extracted features comprise matrices of different sizes that can capture different representations of the image, such as specific shapes that are particular to the desired output. The extracted features 410 are input into a decision maker 412 (e.g., a segmentation model), which analyzes the extracted features and outputs one or more predicted polygons 414. Note that a segmentation model is a computation model (e.g., a machine learning model) that divides (e.g., segments) an image into different regions. Some segmentation models described herein assign an integer to each portion (e.g., pixel) of an image, wherein the integer represents a class of the segmentation to which that pixel belongs.

In some embodiments, the feature extractor 408 and the decision maker 412 comprise (e.g., are a part of) a trained segmentation model (e.g., a neural network). The feature extractor 408 is configured to extract pixels from the pre-processed ultrasound images. In some embodiments, the decision maker 412 is configured to classify (e.g., determine) whether a respective pixel (or portion comprising multiple pixels) belongs to one of: a background, a pleura, or an A-line. In some embodiments, the decision maker 412 is configured to output a first value (e.g., a first segmentation value) (e.g., a value of “0”) when the respective pixel (or portion comprising multiple pixels) is determined to be part of a pleura. In some embodiments, the decision maker 412 is configured to output a second value (e.g., a second segmentation value) (e.g., a value of “1”) when the respective pixel is determined to be part of an A-line. In some embodiments, the decision maker 412 is configured to output a third value (e.g., a third segmentation value) (e.g., a value of “2”) when a respective pixel is determined to be part of a background. In some embodiments, the decision maker 412 determines that a respective pixel belongs to the background when it is determined that the pixel is part of a pleura class and not part of an A-line. For example, a pixel corresponding to a rib, or a region beneath a rib, is classified as the background class. In some embodiments, the decision maker 412 is configured to output a confidence score with the segmentation value, the confidence score indicating a degree of confidence of the segmentation value or a region classified as having the segmentation value (e.g., a predicted polygon, as described below).

In some embodiments, the one or more predicted polygons 414 correspond to one or more segmented regions. For example, in some embodiments, the decision maker 412 forms a respective segmented region by grouping together pixels that belong to the same class (e.g., have the same segmentation value) and that are in close proximity (e.g., continuous, adjacent, contiguous, connected) with each other. In some embodiments, a respective polygon (e.g., a segmented region) comprises a group of connected pixels satisfying a threshold number of pixels (e.g., at least three pixels, at least five pixels, or at least 10 pixels). In some embodiments, the polygon is determined so as to smooth the edges of the segmented region.

Referring again to FIG. 4, in some embodiments, the predicted polygons 414 include one or more A-line polygons (e.g., segmented region(s) corresponding to the A-line class), one or more pleura polygons (e.g., segmented region(s) corresponding to the pleura class), and/or one or more background polygons (e.g., segmented region(s) corresponding to the background class). In some embodiments, the A-line polygon(s) and the pleura polygon(s) are input into a pleura/A-line separator 416, which is configured to (1) separate the pleura polygon(s) from the A-line polygon(s) and (2) output A-line polygons 418. The A-line polygons 418 are then input into a box finder 420, which examines a respective image relative to other images of the sequence of images in temporal space to produce A-lines 422 (e.g., actual A-lines).

FIG. 5 illustrates a temporal sequence of ultrasound images of a lung that includes five images 502, 504, 506, 508, and 510. Each of the images 502, 504, 506, 508, and 510 includes the pleura 514, which is the horizontal bright line at the top of the image. Each of the images 502, 504, 506, 508, and 510 also includes a respective predicted (e.g., suggested) A-line polygon 512 (e.g., polygons 512-1 to 512-5). In some embodiments, for a candidate image, the box finder 420 identifies one or more predicted A-line polygons in the candidate image and identifies a predetermined number of consecutive images (e.g., five images, 10 images, or 15 images) that immediately precede and/or immediately follow the candidate image. In some embodiments, the box finder 420 determines with a degree of confidence (e.g., at least 70% confidence, at least 80% confidence) that a predicted A-line polygon in the candidate image corresponds to an actual A-line when each of the consecutive images also includes a respective predicted A-line polygon that is approximately at the location as the predicted A-line polygon in the candidate image. Referring again to FIG. 5, and using the image 506 as an example candidate image, in some embodiments, the box finder 420 determines that the predicted A-line polygon corresponds to an actual A-line when (1) each of the images 502, 504, 508, and 510 includes a respective predicted A-line polygon 512 (e.g., polygon 512-1, 512-2, 512-4, and 512-5) that is approximately in the same location as the predicted A-line polygon 512-3, (2) each of the predicted A-line polygons has a length that is approximately equal to the length of the pleura, and/or (3) a respective distance 516 (e.g., 516-1 to 516-5) between each of the predicted polygons 512 and the pleura is approximately the same.

Note that the term “actual A-line,” as used herein, means a region of one or more ultrasound images that the computing device has validated as an A-line (e.g., using results from a segmentation model on the one or more images). The term “candidate A-line,” on the other hand, means the results of the segmentation model, including any post processing steps (e.g., creation of the predicted polygons, as described above) prior to such validation. In some embodiments, a candidate A-line becomes an actual A-line after the candidate A-line is validated. In some embodiments, a plurality of candidate A-lines (e.g., from different images in a sweep) are validated and then averaged to form an actual A-line, such that the actual A-line may not be identical to any of the candidate A-lines.

Although FIG. 5 shows the presence of one A-line in a respective ultrasound image, in some cases, an ultrasound image of a lung can include two or more A-lines, as illustrated in FIG. 6.

In some embodiments, the segmentation model described in FIG. 4 is trained by feeding a large number of ultrasound images (e.g., 1000 images, 10,000 images, 100,000 images, or one million images) through a model that is randomly initialized, getting the output of the model and comparing it to the manually annotated (segmented by an annotator using annotation tools) masks containing the true polygons for A-lines and pleura. By comparing the two, the model can calculate the difference between the two (which would be the error of prediction) and based on that, update the parameters of the decision maker and feature extractor to make the model predict masks that are closer to the true, manually annotated mask.

FIG. 7 illustrates a workflow 700 for detecting B-lines in ultrasound images of a lung, in accordance with some embodiments. In some embodiments, the workflow 700 is performed by one or more processors (e.g., CPU(s) 302) of a computing device that is communicatively connected with an ultrasound probe. For example, in some embodiments, the computing device is a server or control console (e.g., a server, a standalone computer, a workstation, a smart phone, a tablet device, a medical system) that is in communication with the ultrasound probe. In some embodiments, the computing device is a control unit integrated into the ultrasound probe. In some embodiments, the ultrasound probe is a handheld ultrasound probe or an ultrasound scanning system.

The workflow 700 includes obtaining (e.g., receiving) input data 702. In some embodiments, the workflow 400 is performed without user intervention in response to receiving the input data 402. In some embodiments, the data 702 comprises a series of lung ultrasound images (e.g., an image stack, a sweep) that are acquired during a scan (e.g., a single scan, a continuous scan, etc.). In some embodiments, the data 702 is input into an A-line detector 704 that is configured to determine (e.g., identify) presence of one or more A-lines and/or pleura, and output one or more A-line masks and/or pleura masks 706 identifying the locations of these features in the image. In some embodiments, the A-line detector 704 comprises a segmentation model. In some embodiments, the A-line detector 704 corresponds to at least a subset of: the feature extractor 408, the decision maker 412, and the pleura/A-line separator 416 that are described in FIG. 4, and not repeated for the sake of brevity.

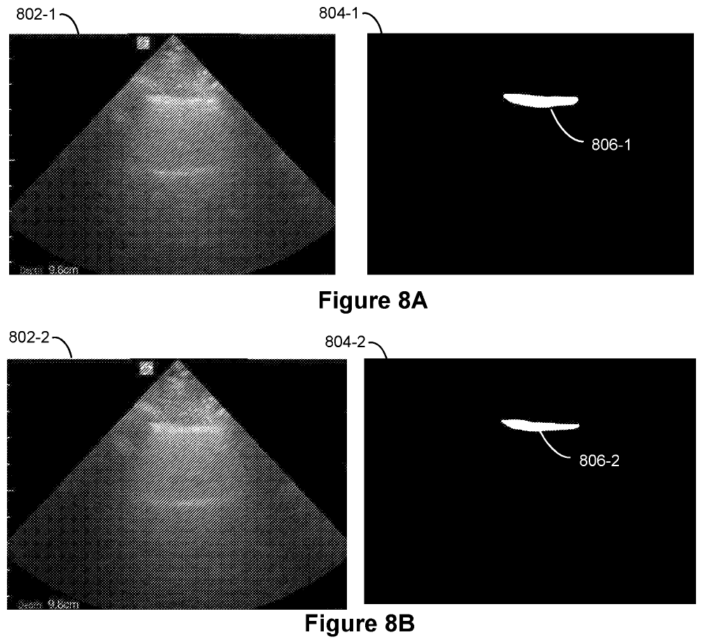

FIGS. 8A and 8B illustrate, on the left, exemplary ultrasound images 802-1 and 802-2 that are input into the A-line detector 704. The right images 804-1 and 804-2 in FIGS. 8A and 8B show respective pleura masks 806-1 and 806-2 that are output by the A-line detector 704.

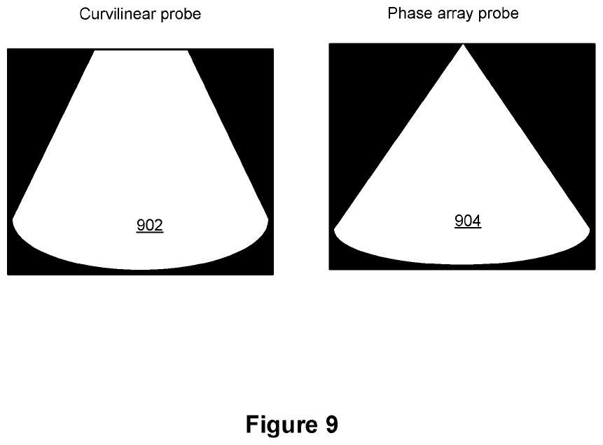

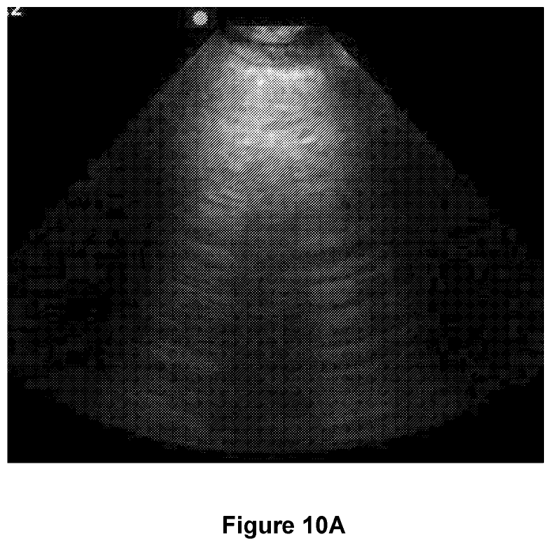

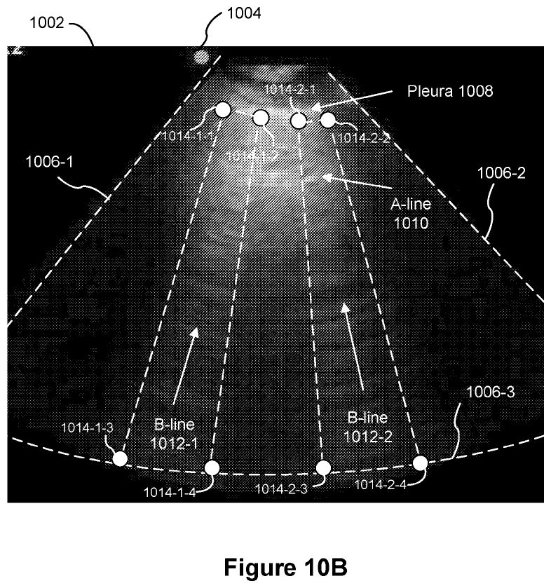

In some instances, depending on the type of probe (transducer) that is used, the ultrasound image can have a field of view that has a wedge shape, a cone shape, or a parallel shape. For example, FIG. 9 shows that a curvilinear probe can provide a field of view 902 that has a wedge shape, whereas a phase array probe can provide a field of view 904 that has a cone shape, in accordance with some embodiments. FIGS. 10A and 10B show the same exemplary ultrasound image 1002 that is acquired using a curvilinear probe. FIG. 10A shows the ultrasound image 1002 as-acquired. FIG. 10B identifies some of the features in the ultrasound image 1002. In FIG. 10B, the ultrasound image 1002 includes an indicator 1004 (e.g., an orientation marker). The ultrasound image 1002 has a wedge shape field of view, defined (e.g., partially) by line 1006-1, line 1006-2, and arc 1006-3. The ultrasound image 1002 also shows the presence of a pleura 1008, a possible A-line 1010, and B-lines 1012 (e.g., B-line 1012-1 and B-line 1012). In some embodiments, a respective B-line 1012 is defined by its four vertices (e.g., corners) 1014-1, 1014-2, 1014-3, and 1014-3. For example, B-line 1012-1 includes vertices 1014-1-1 to 1014-1-4, whereas B-line 1012-2 includes vertices 114-2-1 to 1014-1-4.

Referring again to the workflow 700 in FIG. 7, in some embodiments, the data 702 (e.g., a series of ultrasound images) are input into a cone-to-rectangle converter 708, which is configured to convert a field of view of a respective image from a cone/wedge shape to a rectilinear (e.g., rectangle) shape.

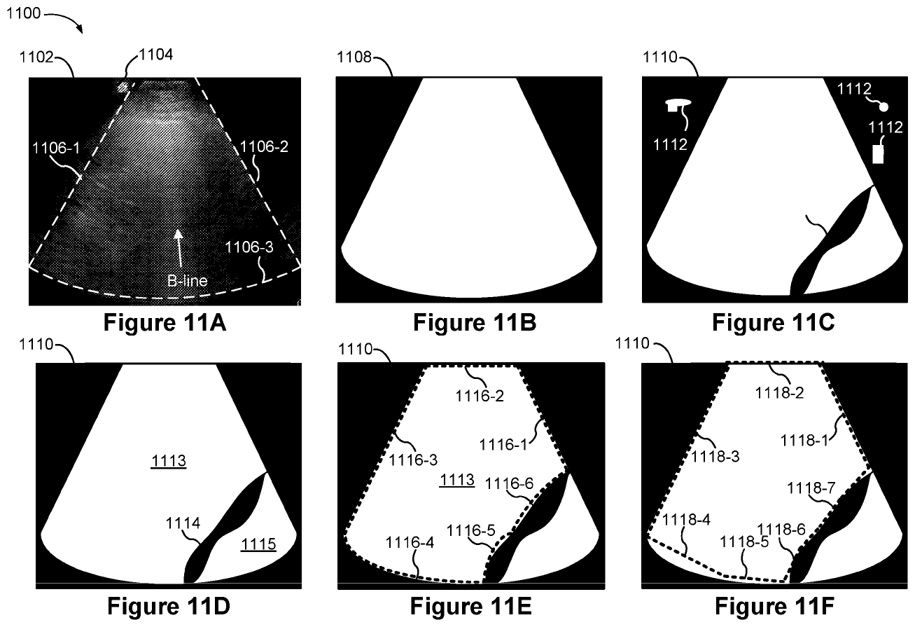

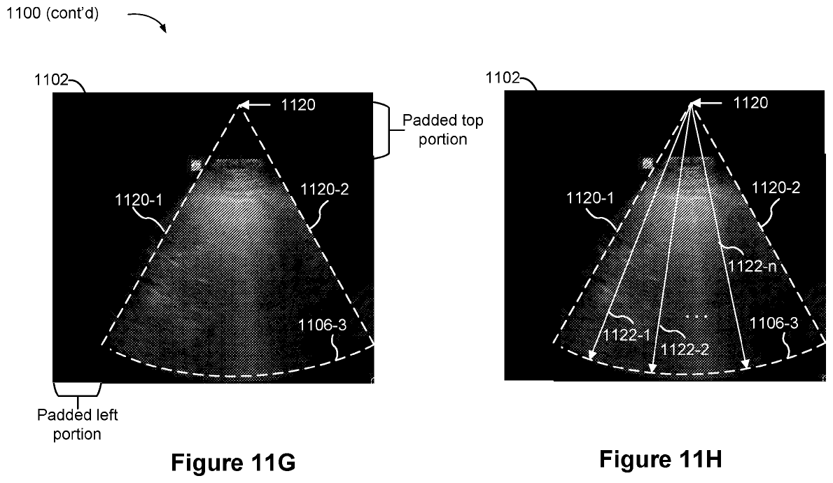

FIGS. 11A and 11I illustrate an exemplary process 1100, performed by the cone-to-rectangle converter 708, for converting a field of view from a cone/wedge shape to a rectilinear shape, in accordance with some embodiments. To do so, exemplary process 1100 estimates lines 1106-1 and 1106-2 through a number of steps, described below. The estimated lines (shown as lines 850-1 and 850-2 in FIGS. 11G-11F) are then used to convert the field of view from a cone/wedge shape to a rectilinear shape.

FIG. 11A shows an ultrasound image 1102 that includes a cone/wedge shape field of view that is defined by the lines 1106-1, 1106-2, and 1106-3. In some embodiments, the ultrasound image 1102 is an image in a series of ultrasound images (e.g., a sweep), and the cone-to-rectangle converter 708 is configured to perform the process 1100 on every ultrasound image in the series of ultrasound images. In some embodiments, the ultrasound image 1102 is an image in a series of ultrasound images, and the cone-to-rectangle converter 708 is configured to perform the process 1100 on a subset of images in the series.

In some embodiments, the process 1100 includes a statistical analysis of the pixels in two or more images in the series of images (e.g., to determine which parts of the images are static and thus unlikely to represent actual ultrasound data). For example, the statistical analysis may be a standard deviation computation step, wherein the cone-to-rectangle converter 708 determines a standard deviation for the intensity of each pixel in a respective ultrasound image, across all the images in the image stack (e.g., series of ultrasound images). Using the image 1102 in FIG. 11A as an example, pixels that are within the area defined by the lines 1106-1 and 1106-2 will have a non-zero standard deviation value, whereas pixels outside the area defined by the lines 1106-1 and 1106-2 will have a standard deviation value of zero. FIG. 11A shows that the image 1102 includes an indicator 1104 (e.g., an orientation marker). Pixels corresponding to the indicator 1104 will have a standard deviation value of zero, because the indicator 1104 appears at the same position for every image in the series. Thus, the statistical analysis provides a device-agnostic way to determine the region of actual ultrasound data in a series of images, thus allowing the methods described herein to be used with a variety of ultrasound devices.

FIG. 11B shows a binary mask 1108 that is generated after the standard deviation computation step, in accordance with some embodiments. A pixel in the binary mask 1108 is assigned a value of “0” (e.g., a black pixel) when the corresponding pixel in the ultrasound image stack has a standard deviation value of zero (e.g., determined in the standard deviation computation step). A pixel in the binary mask 1108 is assigned a value of “255” (e.g., a white pixel) when the corresponding pixel in the image stack has a standard deviation value that is non-zero (e.g., determined in the standard deviation computation step). The binary mask 1108 that is shown in FIG. 11B represents an ideal scenario where there is no noise in the image stack. FIG. 11C illustrates a binary mask 1110 that is generated after the standard deviation computation step, when noise is present in the image stack. FIG. 11C shows that in some instances, the presence of noise in the images can result in one or more white noise regions 1112 appearing in the areas outside the cone/wedge. In some instances, the presence of noise in the images can result in one or more black regions 1114 appearing in the areas within the cone/wedge.

In some embodiments, the process 1100 includes applying a binary opening operation (e.g., a morphological operation that includes an erosion and a dilation) to a binary mask 1110 to remove one or more white noise regions 1112 from the binary mask 1110. FIG. 11D shows an updated view of the binary mask 1110, in which the white noise regions 1112 have been removed, after the binary opening operation has been applied.

In some embodiments, the process 1100 includes determining the biggest connected component (e.g., region) in the binary mask. FIG. 11D shows that the presence of the black region 1114 “splits” the cone/wedge area of the binary mask 1110 into a first area 1113 and a second area 1115. In this example, the biggest connected component (e.g., region) in the binary mask 1110 corresponds to the first area 1113, defined by the boundaries 1116-1 to 1116-6, as illustrated in FIG. 11E.

In some embodiments, the process 1100 includes determining the convex hull of the biggest connected component. In some embodiments, this step comprises fitting a polygon to the biggest connected component. FIG. 11F illustrates determining the convex hull of the area 1113 by fitting a polygon, defined by lines 1118-1 to 1118-7, to the area.

In some embodiments, after finding the convex hull, the cone-to-rectangle converter 708 applies a Hough line transform to determine lines of the convex hull and their corresponding angles. In some embodiments, the cone-to-rectangle converter 708 filters the lines by only keeping those that fall within a certain range of distances (e.g., within the width of the ultrasound image) and/or angles (e.g., between 40° and 140°, or between 45° and 135°). The cone-to-rectangle converter 708 estimates two lines 1120-1 and 1120-2 corresponding to the sides of the cone (e.g., corresponding to the lines 1106-1 and 1106-2 in FIG. 11A) by determining an average for the filtered lines.

In some embodiments, the process 830 includes padding the top and left portions of the image 1102 with black pixels so as to create enough space for the resultant rectangle (e.g., rectilinear) frame. FIG. 11G illustrates the ultrasound image 1102 after additional black pixels have been added to the top and left portions of the image and a point of intersection 1120 between the estimated lines 1120-1 and the line 1120-2 has been determined. The process 1100 then creates a range of angles between the two lines 1120-1 and the line 1120-2, with a predetermined step size (e.g., a range of angles from 45° to 135°, with a step size of 0.5°). At each predetermined step size, the process 1100 creates a respective vector 1122 (e.g., vectors 1122-1 to 1122-n in FIG. 11H). A respective vector 1122 comprises the pixel values of the overlap between the vector 1122 and the ultrasound image 1102. The process 1100 includes, for a respective vector 1122, apply a transformation 1124 to the respective vector 1122 that would make the respective vector 1122 vertical, and then applying those pixel values in the respective vector 1122 to a respective new vertical line 1126 (e.g., vertical lines 1126-1 to 1126-n), as illustrated in FIG. 11I. FIG. 11I shows that the new vertical lines are included within a rectilinear frame 1128.

With continued reference to the workflow 700 in FIG. 7, in some embodiments, after transforming the pixels to the rectilinear frame 1128, as described with respect to FIGS. 11A to 11I. a rectangle image 710 is formed (see, e.g., FIG. 11I). The workflow 700 includes applying a horizontal analyzer 712 to analyze intensities corresponding to the pixel columns of a respective rectangle image 710 (e.g., across the transformed image stack). The horizontal analyzer 712 determines presence of one or more B-lines (e.g., B-line rectangle masks 714) according to difference(s) in pixel intensity values for respective columns in a respective image.

FIG. 7 also illustrates that in some embodiments, the A-line masks and/or pleura masks 706 (e.g., pleura masks 806-1 and 806-2 in FIGS. 8A and 8B) that are output by the A-line detector 704 also undergo the cone/wedge shape to rectilinear shape transformation process 830 as described above with respect to FIGS. 11A to 11I, to transform the A-line/pleura masks 706 from a cone/wedge field of view to a rectilinear (e.g., rectangle) field of view (A-line/pleura rectangle masks 716).

In some embodiments, the workflow 700 inputs the B-line rectangle masks 714 and the A-line/pleura rectangle masks 716 into a pleura line filter 718, to generate a B-line rectangle masks subset 720 (e.g., actual B-lines). Because a B-line should start from the pleura and extend below the pleura (see FIGS. 10A and 10B for an ultrasound image that includes the pleura 1008 and B-lines 1012), in some embodiments the pleura line filter 718 overlays the pleura rectangle masks 716 and the B-line rectangle masks 714, identifies B-lines that are beneath the pleura as actual B-lines, and discards (candidate) B-lines that are above the pleura.

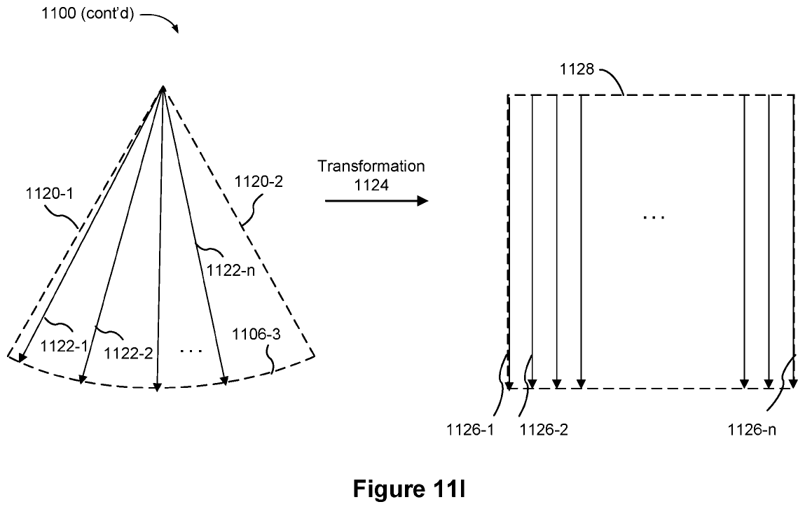

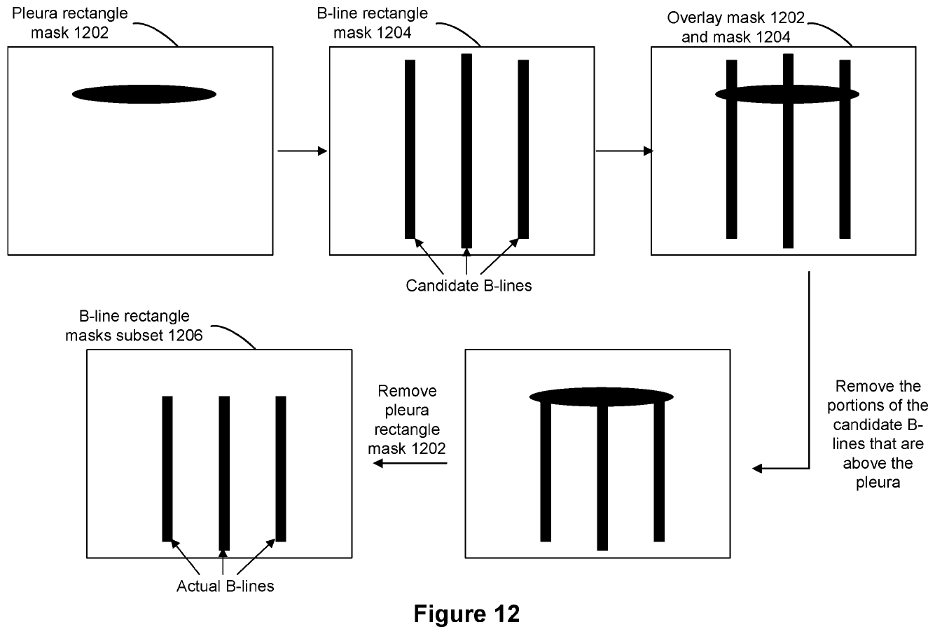

FIG. 12 illustrates a process for obtaining actual B-lines, performed by a pleura line filter 718, in accordance with some embodiments. The pleura line filter 718 receives a pleura rectangle mask 1202 showing the location of a pleura in an image. The pleura line filter 718 receives a B-line rectangle mask 1204 that includes three candidate B-lines. The pleura line filter 718 overlays the pleura rectangle mask 1202 and the B-line rectangle mask 1204, and removes the portions of the candidate B-lines that are above the pleura. The pleura line filter 718 removes the pleura mask 1202 to obtain a B-line rectangle masks subset 1206 (e.g. actual B-lines in the rectangle coordinate system).

With continued reference to FIG. 7, the B-line rectangle masks subset 720 is then input into a B-line locator 722, which determines the start and end coordinates (B-line coordinates 724) for a respective B-line in the subset 720. In some embodiments, the workflow 700 includes applying a rectangle-to-cone converter 726 (e.g., a reverse transformation process), which transforms the B-lines from the rectilinear frame (e.g., coordinate system) to the cone/wedge coordinate system, to obtain cone B-lines 728 (e.g., which can then be displayed to the user (e.g., the operator of the ultrasound probe)).

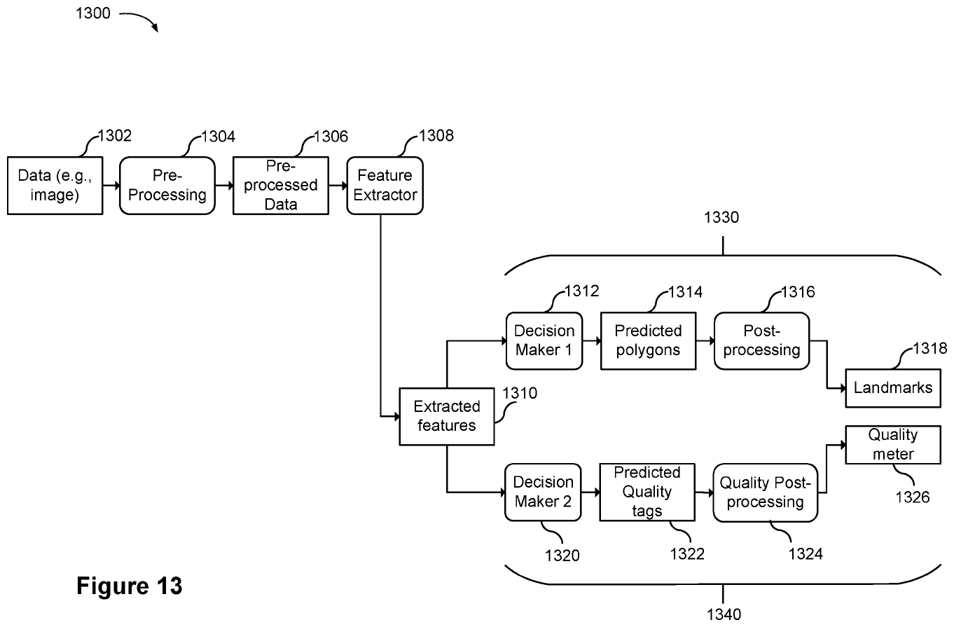

FIG. 13 illustrates a workflow 1300 for acquiring ultrasound scan data, in accordance with some embodiments. In some embodiments, the workflow 1300 is performed by one or more processors (e.g., CPU(s) 302) of a computing device that is communicatively connected with an ultrasound device (e.g., ultrasound device 200). For example, in some embodiments, the computing device is a server or control console (e.g., a server, a standalone computer, a workstation, a smart phone, a tablet device, a medical system) that is in communication with the ultrasound probe. In some embodiments, the computing device is a control unit integrated into the ultrasound device. In some embodiments, the ultrasound device is a handheld ultrasound device or an ultrasound scanning system.

The workflow 1300 includes obtaining (e.g., receiving) data 1302 (e.g., ultrasound images, image stack) from an ultrasound device. In some embodiments, the data 1302 comprises ultrasound data of a lung. In some embodiments, the computing device pre-processes the data 1302 via a pre-processing step 1304 to obtain pre-processed data 1306. In some embodiments, the pre-processing includes normalizing respective intensities of the ultrasound images. In some embodiments, the pre-processing includes adjusting a respective image size of the ultrasound images. In some embodiments, the pre-processed data 1306 comprises lung ultrasound images in which the image intensities have been normalized and/or the image sizes have been adjusted. The pre-processed data 1306 is then input into a feature extractor 1308, which generates extracted features 1310.

In some embodiments, the extracted features 1310 are input into a multi-headed model. The multi-headed model comprises a trained model that is configured to concurrently perform image segmentation and image classification. The image segmentation produces one or more segmented regions (e.g., A-line region(s), B-line region(s), pleura, and/or background) corresponding to an ultrasound image. The image classification classifies the image as one of: good quality, moderate quality, or poor quality.

In some embodiments, the multi-headed model includes a first head 1330 (e.g., a segmentation portion) that is configured to output one or more landmarks 1318 (e.g., segmented region(s)) for a respective ultrasound image (or for the series of ultrasound images). The first head 1330 includes a decision maker 1312 (Decision Maker 1) that is configured to output one or more predicted polygons 1314. In some embodiments, the decision maker 1312 corresponds to the decision maker 412 as described in FIG. 4. In some embodiments, the predicted polygons 1314 correspond to the predicted polygons 414 in FIG. 4. In some embodiments, the predicted polygons 1314 comprise one or more segmented regions, including A-line region(s), a pleura region, one or more ribs adjacent to the pleura region, and/or background region(s). In some embodiments, the landmarks 1318 are concurrently displayed (e.g., as images are being acquired) with the respective image (e.g., as an overlay over the respective image) or with an image obtained in close temporal proximity to the respective image (e.g., as images are acquired, the computing device continuously determines landmarks using acquired images and displays those landmarks on subsequent images for a predetermined number of images or a predetermined amount of time (e.g., 0.5 seconds, 1 second)). In some embodiments, the respective image (or the series of images) undergoes one or more post-processing steps 1316 (e.g., image smoothing, image sharpening, noise reduction, etc.) prior to being displayed with the landmarks 1318.

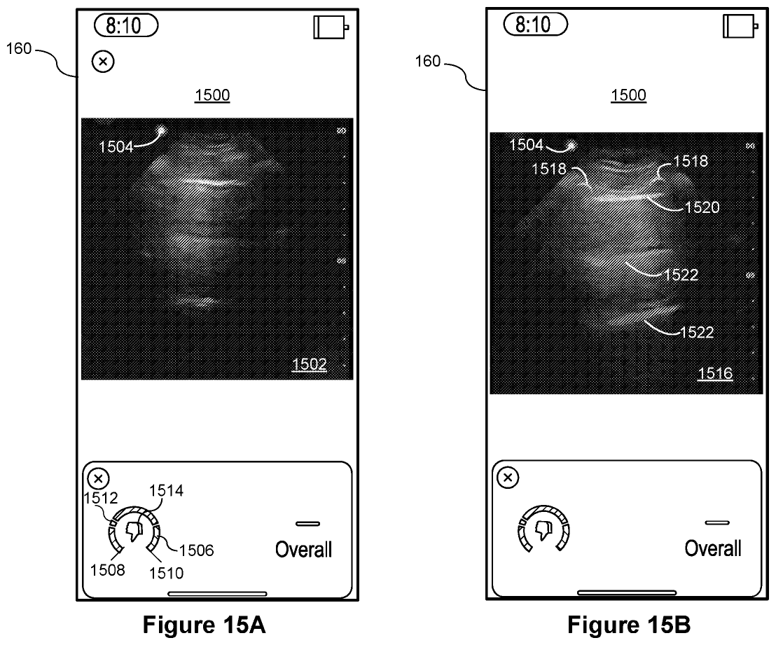

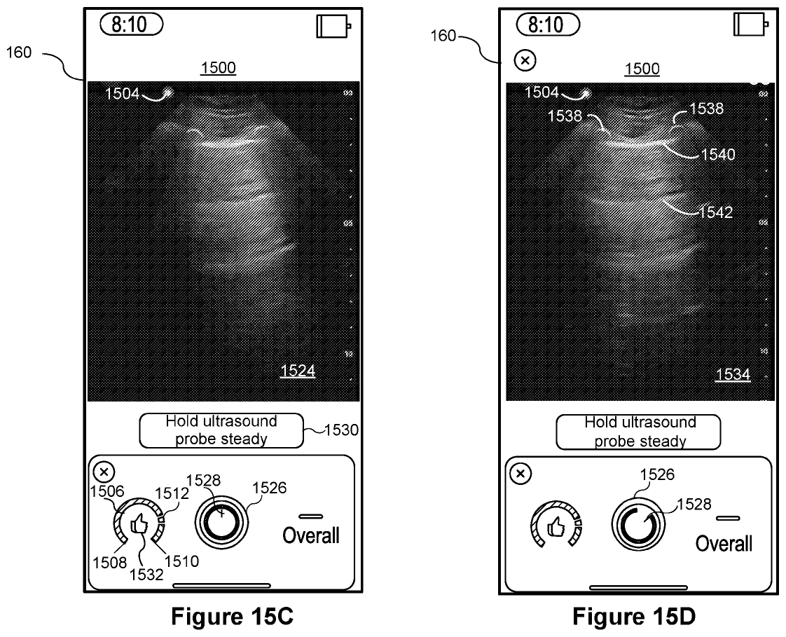

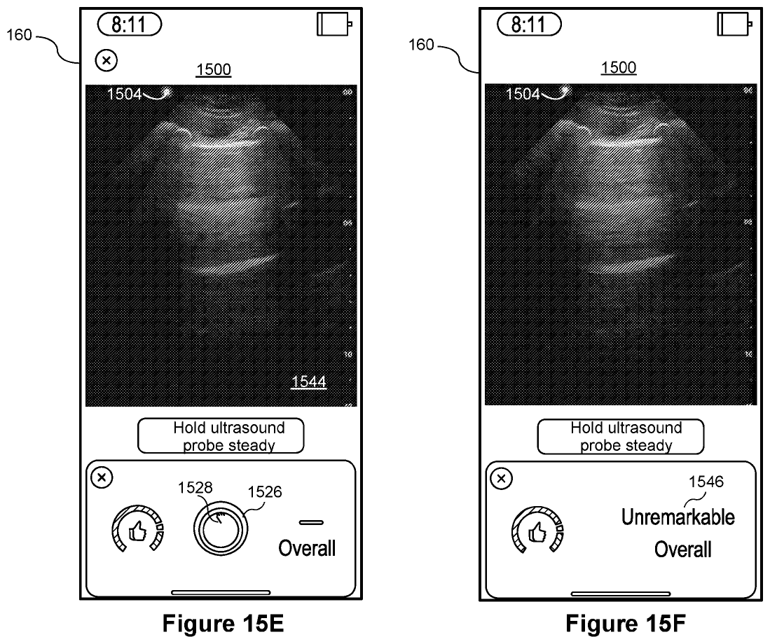

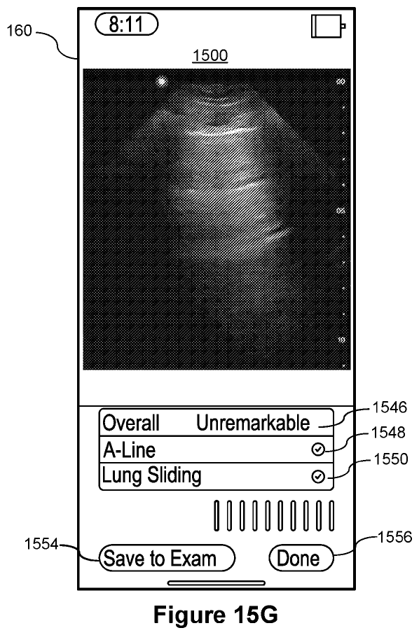

FIG. 13 also illustrates that, in some embodiments, the multi-headed model includes a second head 1340 (e.g., a classification portion) that is configured to output a quality meter 1326 (e.g., quality metric or quality score) for a respective ultrasound image (or for the series of ultrasound images). The second head 1340 includes a decision maker 1320 (Decision Maker 2) that is configured to output a respective predicted quality tag for a respective ultrasound image of the image dataset. In some embodiments, the decision maker assigns, to a respective image, a value of “0,” “1,” or “2,” corresponding to a determination that the respective image is of poor quality, moderate quality, or high quality, respectively. The decision maker 1320 then performs a weighted average across all the images in the dataset to obtain a quality meter 1326. In some embodiments, the quality meter 1326 is a number between zero and one. In some embodiments, the quality meter 1326 is concurrently displayed with the respective image that includes the landmarks 1318, as illustrated with respective to FIGS. 15A to 15G. In some embodiments, the predicted quality tags 1322 undergo a quality post-processing step 1324 prior to being output as the quality meter 1326. For example, the output of the classification head (second head 1340) provides a probability for each of the outcomes (e.g., 0, 1, or 2) and the post-processing performs the following calculation:

$$Q = ( 0 * ( P 0 ) + 1 * ( P 1 ) + 2 * ( P 2 ) ) / 2 \qquad ( 1 )$$

wherein P0 denotes a probability for outcome 0, P1 denotes a probability for outcome 1, P2 denotes a probability for outcome 2, and P0+P1+P2=1. Stated another way, equation (1) multiplies each outcome value by its corresponding probability and then divides by 2, which will give a number Q between 0 and 1. The number Q can be shown to the user as a quality meter.

In some embodiments, the first head 1330 and the second head 1340 are jointly trained (e.g., using labeled data), which results in better performance of both the first head 1330 and the second head 1340.

In some embodiments, the landmarks 1318 and/or the quality meter 1326 are displayed (or caused to be displayed) in real time (or near real time) during an ultrasound scan, providing real time feedback to a user of the ultrasound device 200 and/or assisting the user to obtain high-quality images that contains the features of interest. This is illustrated in FIGS. 15A to 15G. For example, in some instances, in response to observing the landmarks 1318 and/or the quality meter 1326, the user can use an adjusted set of scan parameters to acquire another set of ultrasound images, which in turn generate updated views of the landmarks 1318 and/or quality meters 1326. Thus, the landmarks 1318 and/or the quality meter 1326 inform the user about how the adjusted set of scan parameters affects image quality.

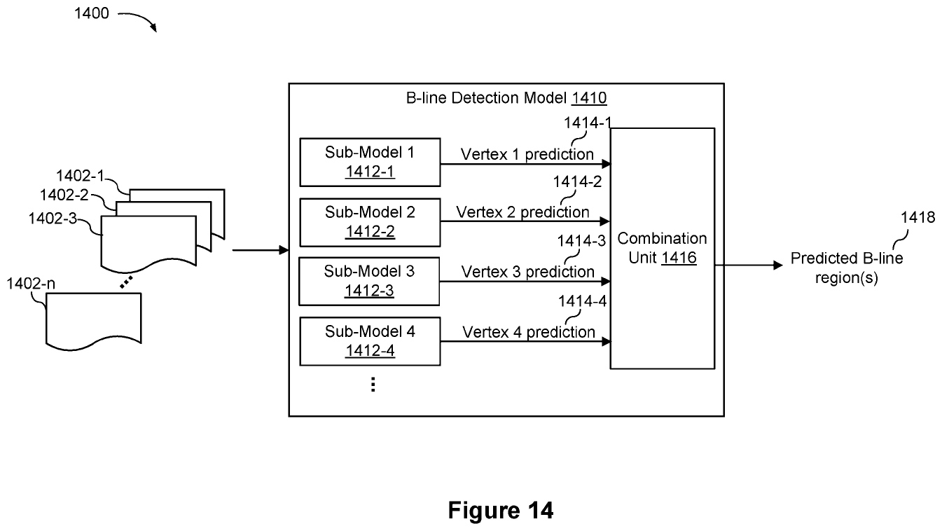

FIG. 14 illustrates an exemplary process 1400 for detecting B-lines (e.g., B-lines 1012) from lung ultrasound images, in accordance with some embodiments. The process 1400 includes inputting lung ultrasound images 1402 into a B-line detection model 1410 that is configured to determine (e.g., predict) one or more B-line regions 1418 from a respective lung ultrasound image 1402.

In some embodiments, the B-line detection model 1410 predicts four distinct outputs, each corresponding to a corner of a trapezoid that defines a potential B-line. Each of the four outputs is in the form of a distinct segmentation mask (a 2D image, with the same height and width of the original ultrasound image), where the value of each pixel represents the probability of that particular pixel belonging to the area of interest, which in this case would be one of the corners of that trapezoid. As a more specific example, B-line detection model 1410 outputs a first segmentation mask that corresponds to one or more top left corners of one or more B-lines, respectively; a second segmentation mask that corresponds to one or more top right corners of the one or more B-lines, respectively; a third segmentation mask that corresponds to one or more bottom left corners of the one or more B-lines, respectively; and a fourth segmentation mask that corresponds to one or more bottom right corners of the one or more B-lines, respectively.

In some embodiments, a B-line can be approximated as a trapezoidal shape with four corners. Referring to the lung ultrasound image in FIG. 10B, the B-line 1012-1 includes four corners 1014-1-1 to 1014-1-4 to 1012-4 and the B-line 1012-2 includes four corners 1014-2-1 to 1014-2-4. In some embodiments, the B-line detection model 1410 is a pre-trained segmentation model that is configured to output a set of vertices (e.g., points), each corresponding to a respective corner of a respective B-line in the lung ultrasound image. In some embodiments, each vertex corresponds to one pixel in the ultrasound image. In some embodiments, each vertex corresponds to a predefined number of pixels (e.g., a group of two pixels, three pixels, or five pixels) that forms a contiguous region in the lung ultrasound image.

In some embodiments, the B-line detection model 1410 is configured to identify (e.g., determine) one or more pixels in a lung ultrasound image corresponding to a respective corner of a B-line. In some embodiments, the B-line detection model 1410 is configured to output, for each of the identified pixels, a respective probability that it corresponds to a corner of the B-line, and determine the pixel with the highest probability as corresponding to the corner of a B-line.

In some embodiments, the B-line detection model 1410 includes a plurality of sub-models 1412. Each of the sub-models 1412 is configured to output (e.g., predict or determine) a respective (e.g., predefined) vertex of the set of vertices (via vertex predictions 1414). Using the example where a respective B-line can be approximated as a trapezoidal shape with four vertices, in some instances, the B-line detection model 1410 can include four sub-models 1412-1 to 1412-4, each of which is configured to predict a predefined vertex. For instance, sub-model 1 1412-1 can be configured to predict (1414-1) vertex 1, corresponding to the top-left corner of a respective B-line; sub-model 2 1412-2 can be configured to predict (1414-2) vertex 2, corresponding to the top-right corner of the respective B-line; sub-model 3 1412-3 can be configured to predict (1414-3) vertex 3, corresponding to the bottom-left corner of the respective B-line; and sub-model 4 1412-4 can be configured to predict (1414-4) vertex 4, corresponding to the bottom-right corner of the respective B-line.

As disclosed, the B-line detection model 1410 is capable of detecting one or more B-lines that are present in an ultrasound image. Suppose that a certain frame of an ultrasound image has only one B-line. In this case, each model output will only show one corner in the mask, meaning that there will be a single location in the mask that has non-zero values and the rest of the pixels will have either zero or very close to zero values. In some instances, there may be more than one B-line present in an ultrasound mage. Using an example where there are two B-lines present in an ultrasound image, each mask will have two locations with non-zero values, corresponding to two corners, each belonging to a separate B-line. In this case, the B-line detection model 1410 groups the predicted corners into groups of 4 (each from a separate mask) based on their location within the image. An assumption is that these trapezoids do not overlap so it is straight forward to form groups of four corners.

It will be apparent to one of ordinary skill in the art that in some embodiments, a respective B-line can be approximated as a n-sided polygon with n vertices (e.g., n can be an integer that is three or more). Thus, in some instances, the B-line detection model can include n sub-models, each of which is configured to a respective vertex of the n-sided polygon.

With continued reference to FIG. 14, in some embodiments, the B-line detection model 1410 includes a combination unit 1416 that is configured to combine the respective vertex predictions from the sub-models 1414 and generate predicted B-line region(s) 1418.

In some embodiments, the B-line detection model 1410 is a pre-trained model that is configured to detect one or more B-lines from a respective lung ultrasound image 1402 by predicting respective positions (e.g., coordinates) corresponding to the corners (e.g., vertices 1014-1 to 1014-4) of a respective B-line.

In some embodiments, the B-line detection model 1410 is trained by providing appropriate labels which should look like a desired output that the trained B-line detection model 1410 would produce. The labels for training can be created by annotating (e.g., using an annotation tool on a computer device) each B-line in a set of training images with a respective polygon (e.g., a trapezoidal polygon). An annotated polygon is processed by taking the coordinates of each of the four corners of a polygon and creating a 2D Gaussian distribution around each set of coordinates (i.e., four separate Gaussian distributions). This process is repeated for the polygons in the set of training images. This results in a label (in the form of a mask) where all pixels have value zero except for the corner locations, which are defined by the Gaussian distributions. These labels along with the original images are then used to train the model to generate the trained B-line detection model 1410.

FIGS. 15A to 15G illustrate an example user interface 1500 for displaying ultrasound scan data and/or other feedback, in accordance with some embodiments. In some embodiments, the ultrasound device 200 is paired with an external electronic device 160 (e.g., a cellphone, tablet, laptop), which is used for displaying ultrasound scan data and/or other feedback (e.g., instructions or guidance) generated by the ultrasound device 200.

In FIG. 15A, an operator of the ultrasound device 200 initiates an ultrasound scan (e.g., a lung ultrasound scan) of a patient. At the start of the scan, the operator may move the ultrasound device 200 around the patient's body to locate an appropriate position that would provide an optimal view of the anatomy of interest. In some embodiments, FIG. 15A illustrates the user interface 1500 displaying an ultrasound image 1502 in response to the operator moving the ultrasound device 200 around the patient's body during the initial scan. The ultrasound image 1502 includes an orientation marker 1504. In some embodiments, the ultrasound image 1502 that is displayed on the user interface 1500 is continuously updated (e.g., at a rate of 20, 25, or 30 frames per second) as the operator adjusts a position of the ultrasound device 200 relative to the patient's body.

In some embodiments, the user interface 1500 displays a quality gauge 1506 (e.g., a gauge or a dial) concurrently with the ultrasound image and displays an indicator 1512 on the quality gauge and whose position can vary between a minimum position 1508 and a maximum position 1510. In some embodiments, the minimum position 1508 corresponds to a quality score of zero and the maximum position corresponds to a value of one, and therefore the position of the indicator 1512 corresponds to a quality score with a value between zero and one inclusive (and proportional to a distance between the minimum position 1508 and the maximum position 1510 along the quality gauge 1506).

In some embodiments, the position of the indicator 1512 on the quality gauge 1506 reflects a quality score of the ultrasound image that is concurrently displayed on the user interface 1500. In some embodiments, the position of the indicator 1512 reflects a quality score of all the ultrasound data that has been acquired since the scan initiation. In some embodiments, the position of the indicator 1512 reflects a quality score of a subset of all the ultrasound scan data acquired since the scan initiation (e.g., scan data corresponding to the most recent three frames, the most recent five frames, the most recent five seconds of scan data, or the most recent eight seconds of scan data).

In some embodiments, when the electronic device 160 (e.g., via the multi-headed model) determines that the quality score is less than a predetermined threshold quality score, the user interface 1500 displays a visual representation 1514 (e.g., a thumbs-down image) indicating that the quality score is less than the threshold score.

In some embodiments, the gauge 1506 is color-coded and the operator can easily identify, based on the position of the indicator 1512 on the gauge, whether the ultrasound image is of sufficient quality. For example, in some embodiments, the gauge 1506 is has a color scale from red to orange to green. When the position of the indicator is in the red zone, it indicates that the quality score is less than a threshold score; when the indicator is in the orange zone, it indicates that the quality score is hovering at the threshold score, and when the position of the indicator is in the green zone, it indicates that the quality score is above the threshold score.

In some embodiments, the user interface 1500 displays, concurrently with an ultrasound image, one or more segmented regions (e.g., segmented by the multi-headed model in FIG. 13) of the ultrasound image. FIG. 15B shows a lung ultrasound image 1516 with segmented regions 1518, 1520, and 1522 overlaid on top of it. In this example, the segmented region 1520 corresponds to the pleura, the segmented regions 1518 correspond to two ribs right next to the pleura, and the segmented regions 1522 correspond to A-lines.

In FIG. 15C, the electronic device 160 displays an updated lung ultrasound image 1524 and updates the position of the indicator 1512 on the gauge 1506 in accordance with the most recently acquired ultrasound data. When the electronic device 160 (e.g., via the multi-headed model) determines that the quality score of the ultrasound data satisfies (e.g., meets or exceeds) the threshold quality score, the electronic device displays a visual representation 1532 (e.g., a thumbs-up image) indicating that the quality score satisfies the threshold score. In some embodiments, in response to the determination that the quality score satisfies the threshold quality score, the electronic device 160 displays a notification 1530 to the operator to hold the ultrasound device 200 in its current position and activates record button 1526, to initiate recording of a stack (e.g., a series, a predefined number) of ultrasound images.

In some embodiments, as the stack of ultrasound images is being recorded, the user interface 1500 displays, in real-time or near real-time (e.g., within 0.2 sec, 0.5 sec, or 1 sec of data acquisition), ultrasound image(s) that are generated by the ultrasound device 200 as data is being acquired by the ultrasound device 200. In some embodiments, the user interface 1500 displays, concurrently with an ultrasound image, segmented regions corresponding to the ultrasound image that is being displayed. This is illustrated in FIGS. 15D and 15E. In FIG. 15D, the user interface 1500 displays an ultrasound image 1534 and displays segmented regions 1538, 1540, and 1542 corresponding to and overlaid on the image 1534. The segmented regions 1538 correspond to two ribs right next to the pleura, the segmented region 1540 corresponds to the pleura, and the segmented region 1542 corresponds to an A-line.

In some embodiments, as the stack of ultrasound images is being recorded, the user interface 1500 displays a progress of the recording. For example, FIG. 15D shows that the record button 1526 include a progress indicator 1528 that is at the 12 o'clock position at the start of the recording. As the recording progresses, the progress indicator is updated (e.g., via rotation in the clockwise direction) to the 2 o'clock position in FIG. 15D and to the 11 o'clock position in FIG. 15E.

FIG. 15F illustrates that, in some embodiments, upon completing the recording of the stack of ultrasound images, the user interface 1500 omits display of the record button 1526 and indicator 1528, and displays a status 1546 for the scan. For example, in some embodiments, the status 1546 can include a diagnostic conclusion. For example, in some embodiments, the status 1546 can be one of “Unremarkable,” “Healthy,” “Follow-up Recommended,” or “Inconclusive.” In some instances, the total time taken from acquiring the image stack to receiving the status 1546 takes less than one minute.

In some embodiments, the user interface 1500 displays diagnostic information of the lung based on the stack of ultrasound images. For example, as illustrated in FIG. 15G, the diagnostic information can include the status 1546 for the scan, a condition 1548 of the lung as determined by the presence of A-lines in the ultrasound scan data, and/or presence or absence 1550 of lung sliding. In some embodiments, the user interface 1500 also displays an affordance 1554 (e.g., a user-selectable option) to save the diagnostic information and/or the stack of ultrasound images to the patient's examination records, and an affordance 1556 to complete the scan.

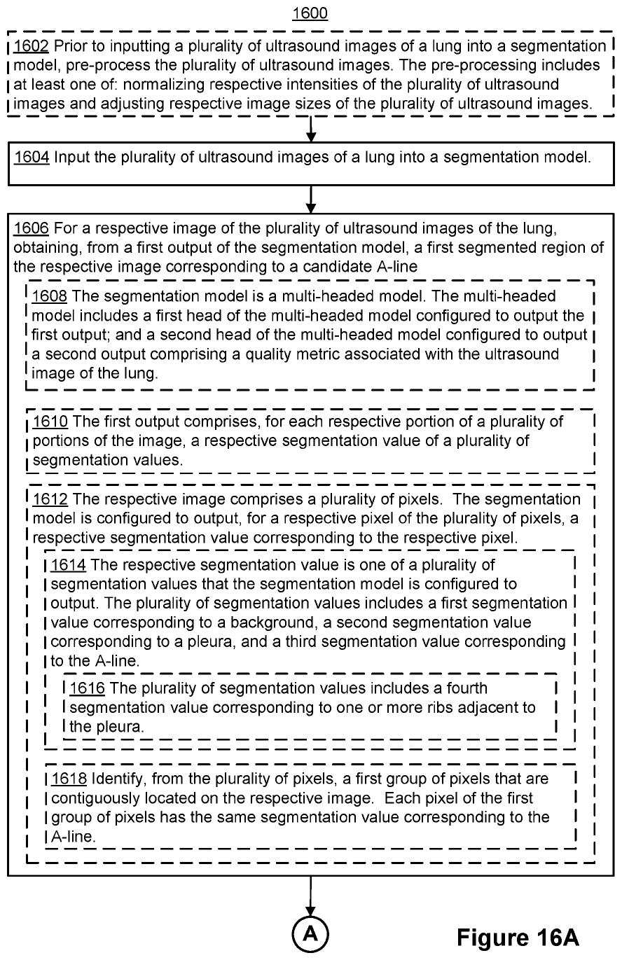

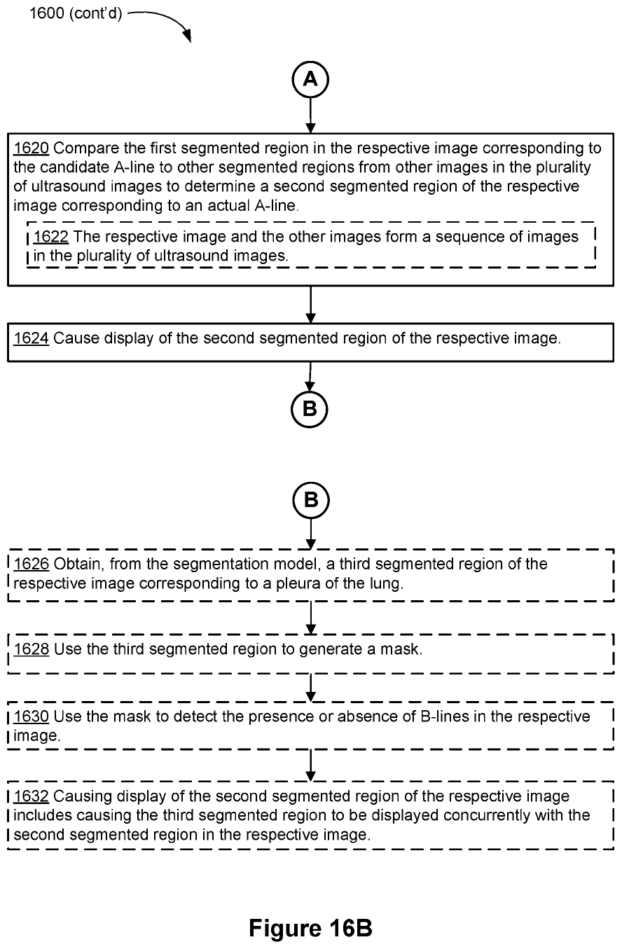

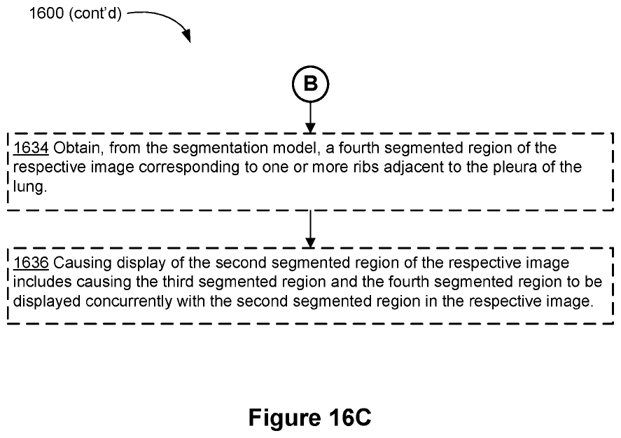

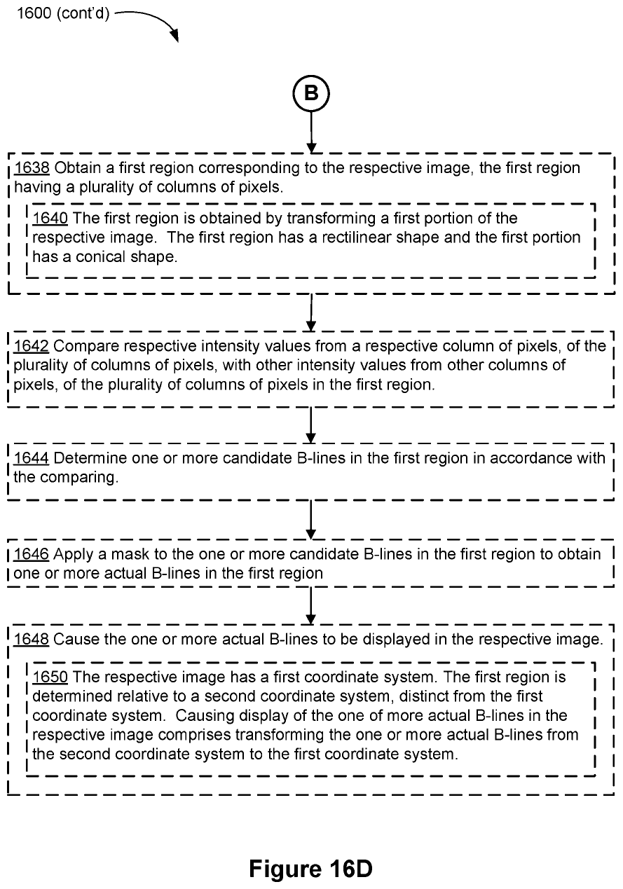

FIGS. 16A to 16D illustrate a flowchart diagram for a method 1600 of detecting the presence of A-lines (e.g., pleural line artifacts) in ultrasound images, in accordance with some embodiments. In some embodiments, the method 1600 is performed at a computing device (e.g., computer system 130 or electronic device 160) that includes one or more processors (e.g., CPU(s) 302) and memory (e.g., memory 306). For example, in some embodiments, the computer device is a server or control console (e.g., a server, a standalone computer, a workstation, a smart phone, a tablet device, a medical system) that is in communication with a handheld ultrasound probe or ultrasound scanning system. In some embodiments, the computing device is a control unit integrated into a handheld ultrasound probe or ultrasound scanning system. In some embodiments, the operations described in method 1600 can be split among different devices (e.g., between a computer system 130 and an ultrasound device 200, between a computer system 130 and an electronic device 160, between an ultrasound device 200 and an electronic device 160, or between multiple computer systems 130).

In some implementations, the operations shown in FIGS. 1 and 4-15G correspond to instructions stored in the memory 306 or other non-transitory computer-readable storage medium. The computer-readable storage medium may include a magnetic or optical disk storage device, solid state storage devices such as Flash memory, or other non-volatile memory device or devices. In some implementations, the instructions stored on the computer-readable storage medium include one or more of: source code, assembly language code, object code, or other instruction format that is interpreted by one or more processors. Some operations in the method 1600 may be combined and/or the order of some operations may be changed. In some implementations, some of the operations in the method 1600 may be combined with other operations in the method 1700, 1800 and/or 1900.

In some embodiments, prior to inputting a plurality of ultrasound images of a lung into the segmentation model, the computer device pre-processes (1602) the plurality of ultrasound images. In some embodiments, the pre-processing includes at least one of: normalizing respective intensities of the plurality of images and adjusting respective image sizes of the plurality of ultrasound images.

The computer device inputs (1604) the plurality of ultrasound images of a lung into a segmentation model (e.g., decision maker 412 in FIG. 4). In some embodiments, the computer device inputs the plurality of ultrasound images into at least two (e.g., distinct) models (e.g., models that perform the workflow 400, workflow 700, workflow 900, process 1400, a model for pleural effusion, and/or a model for consolidation) for processing.

For a respective image of the plurality of ultrasound images of the lung, the computer device obtains (1606), from a first output of the segmentation model, a first segmented region of the respective image corresponding to a candidate A-line.

In some embodiments, the segmentation model is (1608) a multi-headed model. The multi-headed model includes a first head configured to output the first output and includes a head configured to output a second output comprising a quality metric associated with the ultrasound image of the lung. Details of an exemplary multi-headed model are described with respect to FIG. 13.

In some embodiments, the first output comprises (1610), for each respective portion of a plurality of portions of the image, a respective segmentation value of a plurality of segmentation values.

For example, in some embodiments, each portion of the image can correspond to a pixel, or a group of pixels (e.g., five pixels, ten pixels, or twenty pixels), and the first output provides a segmentation value for the pixel or the group of pixels. In some embodiments, the first segmented region corresponds to a region of pixels, each of the pixels in the region having a segmentation value indicative of an A-line. In some embodiments, the first segmentation region is determined by averaging or otherwise smoothing segmentation values in the image.

In some embodiments, the respective image comprises (1612) a plurality of pixels. The segmentation model is configured to output, for a respective pixel of the plurality of pixels, a respective segmentation value corresponding to the respective pixel.

In some embodiments, the respective segmentation value is (1614) one of a plurality of segmentation values that the segmentation model is configured to output, wherein the plurality of segmentation values includes a first segmentation value (e.g., a value 0, 1, or 2; or a value 0, 1, 2, or 3) corresponding to a background, a second segmentation value (e.g., a value 0, 1, or 2; or a value 0, 1, 2, or 3) corresponding to a pleura, and a third segmentation value (e.g., a value 0, 1, or 2; or a value 0, 1, 2, or 3) corresponding to the A-line.

In some embodiments, the plurality of segmentation values includes (1616) a fourth segmentation value (e.g., a value 0, 1, or 2; or a value 0, 1, 2, or 3) corresponding to one or more ribs adjacent to the pleura.

In some embodiments, obtaining the first segmented region of the respective image corresponding to the candidate A-line comprises identifying (1618), from the plurality of pixels, a first group of pixels that are contiguously (e.g., sequentially, continuously) located on the respective image, wherein each pixel of the first subset of pixels has the same segmentation value corresponding to the A-line.

With continued reference to FIG. 16B, in some embodiments, the computer device compares (1620) the first segmented region in the respective image corresponding to the candidate A-line to (e.g., with) other segmented regions from other images in the plurality of images to determine (e.g., via an A-line segmentation model, e.g., a model that performs workflow 400 in FIG. 4) a second segmented region of the respective image corresponding to an actual A-line.

In some embodiments the respective image and the other images form (1622) a sequence of (e.g., consecutive) images in the plurality of images (see, e.g., FIG. 5). For example, in some embodiments, the respective image and the other images are part of a temporal sequence of images (e.g., a temporal sequence of 5 frames, 8 frames, 10 frames, or 15 frames) that are acquired during the same (e.g., a single, continuous, etc.) ultrasound scan.

In some embodiments, the other segmented regions correspond to respective candidate A-lines in the other images. In some embodiments, the computer device compares a location of the first segmented region in the respective image with respective locations of the other segmented regions in the other images, and determines that the second segmented region corresponds to an actual A-line when the first segmented regions and the other segmented regions are located in roughly the same position (e.g., relative to a known anatomical landmark that is present in the temporal sequence of images). In some embodiments, the computer device determines that the candidate A-line in the respective image is an actual A-line when the first segmented region persists in a number of frames (e.g., two frames immediately before the respective frame and three frames immediately after the respective frame, five frames immediately before the respective frame and three frames immediately after the respective frame, etc.) in (e.g., approximately) the same location (e.g., relative to a known anatomical landmark that is present in the temporal sequence of images).

In some embodiments, the computer device compares the first segmented region to the other segmented regions, and determines the second segmented region, corresponding to an actual A-line, according to an extent of overlap (e.g., 70%, 80%, 90%, or 90% overlap) between the first segmented region and the other segmented regions. In some embodiments, a candidate A-line can be ruled out as an actual A-line when it does not meet the requirement for identifying it as an “actual” A-line, for example, when the extent of overlap between the candidate A-line and other candidate A-lines in the other images does not meet a threshold requirement (e.g., less than 85%, less than 90%, less than 95%, etc.).

In some embodiments, the second segmented region partially overlaps (e.g., at least 50% overlap, at least 70% overlap, at least 85% overlap, etc.) with the first segmented region. In some embodiments, the second segmented region is the same region as (e.g., fully overlaps with) the first segmented region. For example, in some embodiments, in accordance with a determination that the comparison of the first segmented region in the respective image to other segmented regions from other images meets one or more consistency criteria, the computing device identifies the first segmented region as an actual A-line region and causes the first segmented region to be displayed. In some embodiments, the second segmented region is an average (or weighted average) of a plurality of segmented regions that, together, meet one or more consistency criteria.

In some embodiments, the computer device causes (1624) display of the second segmented region of the respective image. For example, in some embodiments, the second segmented region is displayed as an overlay of the respective image (see, e.g., FIGS. 15B and 15D), or displayed as an overlay of a representative image of the plurality of ultrasound images.

In some embodiments, the computing device obtains (1626), from the segmentation model, a third segmented region of the respective image corresponding to the pleura of the lung. The computing device uses (1628) the third segmented region to generate a mask (e.g., a pleura mask) (e.g., pleura mask 806-1 in FIG. 8A and pleura mask 806-2 in FIG. 8B). In some embodiments, the computing device uses (1630) the mask to detect the presence or absence of B-lines in the respective image (e.g., using the process as described in the workflow 700).

In some embodiments, causing display of the second segmented region of the respective image includes causing (1632) the third segmented region corresponding to the pleura of the lung to be displayed concurrently with the second segmented region corresponding to the actual A-line in the respective image. This is illustrated in, for example, FIGS. 15B and 15D.

Referring to FIG. 16B, in some embodiments, the computing device obtains (1634), from the segmentation model, a fourth segmented region of the respective image corresponding to one or more ribs adjacent to the pleura of the lung.

In some embodiments, causing display of the second segmented region of the respective image includes causing (1636) the third segmented region corresponding to the pleura of the lung and the fourth segmented region corresponding to one or more ribs adjacent to the pleura of the lung to be displayed concurrently with the second segmented region in the respective image (see, e.g., FIGS. 15B and 15D). In some embodiments, each of the segmented regions is displayed with a respective distinct visual characteristic (e.g., a respective color, line thickness, or text encoding).

With continued reference to FIG. 16D, in some embodiments, the computer device obtains (1638) a first region corresponding to the respective image, the first region having a plurality of columns of pixels.

In some embodiments, the first region is obtained by transforming (1640) (e.g., converting) a first portion of the respective image (e.g., via the transformation process 1124). The first region has a rectilinear shape and the first portion has a conical shape (e.g., cone shape or trapezoidal shape).

In some embodiments, the computer device compares (1642) respective intensity values (e.g., pixel intensity values, luminance values) from a respective column of pixels, of the plurality of columns of pixels, with other intensity values from other columns of pixels, of the plurality of columns of pixels in the first region. The computer device determines (1644) one or more candidate B-lines in the first region in accordance with the comparing.

In some embodiments, the computer device applies (1646) a mask (e.g., A-line/Pleura rectangle masks 716) to the one or more candidate B-lines in the first region (e.g., via the pleura line filter 718) to obtain one or more actual B-lines in the first region. Noting that a B-line should start from the pleura and extend all the way down in the image, in some embodiments, the pleura line filter 718 looks for the presence of B-lines within a mask defined by the pleura (e.g., the region beneath the pleura), and discards anything outside of this region. This is also illustrated in FIG. 12.

In some embodiments, the computer device causes (1648) the one or more actual B-lines to be displayed in the respective image.

In some embodiments, the respective image has (1650) a first coordinate system. The first region is determined relative to a second coordinate system, distinct from the first coordinate system. Causing display of the one or more actual B-lines in the respective image comprises transforming the one or more actual B-lines from the second coordinate system to the first coordinate system.

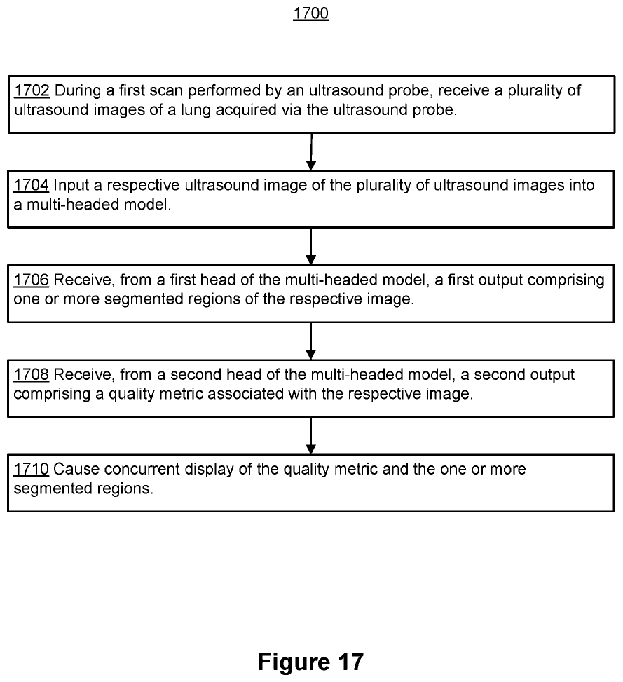

FIG. 17 illustrates a flowchart diagram for a method 1700 of determining scan quality of lung ultrasound images, in accordance with some embodiments. In some embodiments, the method 1700 is performed at a computing device (e.g., computer system 130 or electronic device 160) that includes one or more processors (e.g., CPU(s) 302) and memory (e.g., memory 306). For example, in some embodiments, the computer device is a server or control console (e.g., a server, a standalone computer, a workstation, a smart phone, a tablet device, a medical system) that is in communication with a handheld ultrasound probe or ultrasound scanning system. In some embodiments, the computing device is a control unit integrated into a handheld ultrasound probe or ultrasound scanning system. In some embodiments, the operations described in method 1700 can be split among different devices (e.g., between a computer system 130 and an ultrasound device 200, between a computer system 130 and an electronic device 160, between an ultrasound device 200 and an electronic device 160, or between multiple computer systems 130). Additional details of the method 1700 can be found in FIGS. 1 to 16D and the accompanying descriptions, and are not repeated for the sake of brevity.

During a first scan performed by an ultrasound probe, the computer device receives (1702) (e.g., in real time) a plurality of ultrasound images of a lung acquired via the ultrasound probe. In some embodiments, the ultrasound probe comprises a handheld ultrasound probe or an ultrasound scanner. The ultrasound probe is communicatively connected to the computing device.

The computer device inputs (1704) a respective ultrasound image of the plurality of ultrasound images into a multi-headed model.

The computer device receives (1706), from a first head of the multi-headed model (e.g., first head 1330, FIG. 13) (e.g., segmentation portion), a first output comprising one or more segmented regions of the respective image.

The computer device receives (1708), from a second head of the multi-headed model (e.g., second head 1340, FIG. 13) (e.g., classification portion), a second output comprising a quality metric associated with the respective image.

The computer device causes (1710) concurrent display of the quality metric and the one or more segmented regions.

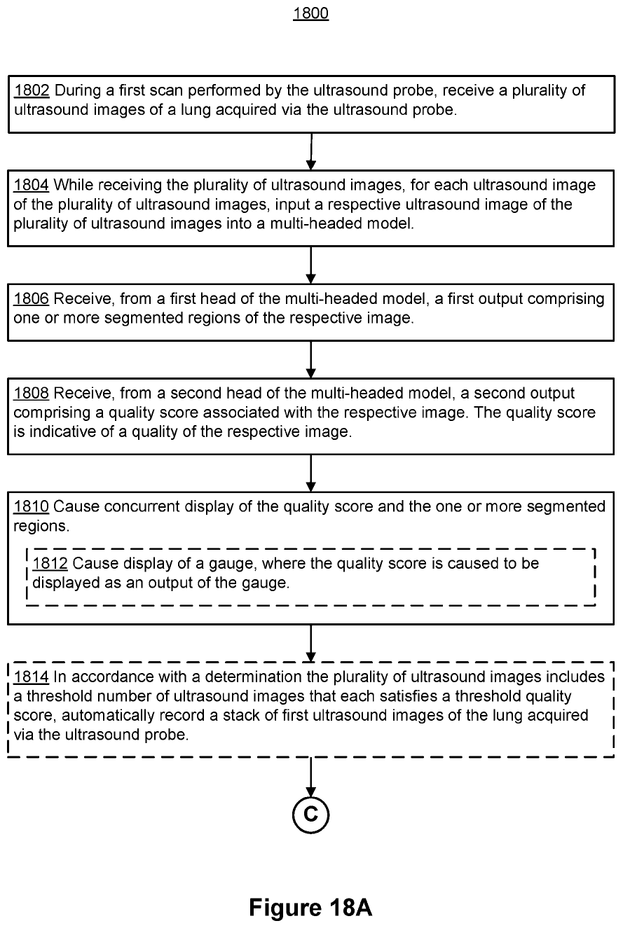

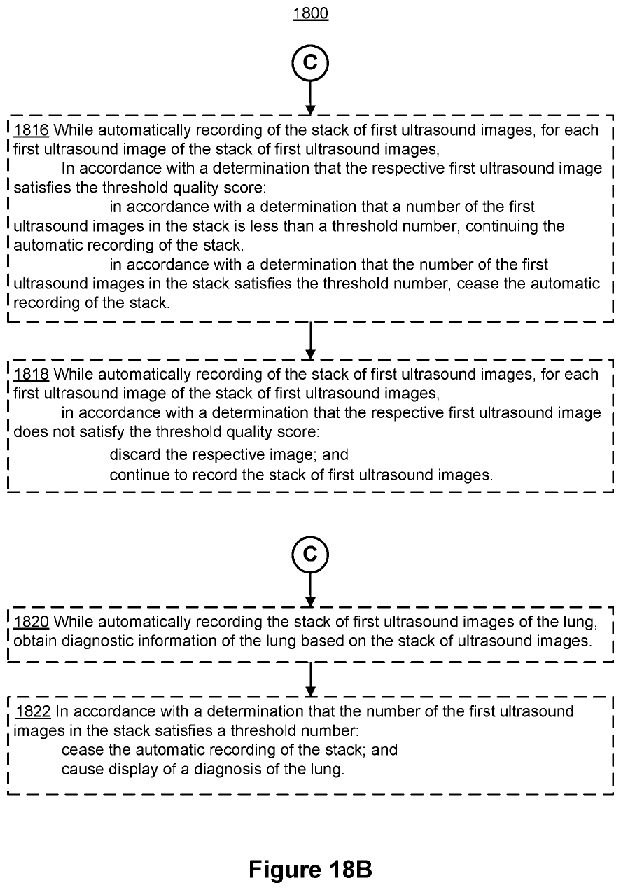

FIGS. 18A and 18B illustrate a flowchart diagram for a method 1800 of determining scan quality of lung ultrasound images acquired via an ultrasound probe (e.g., ultrasound device 200), in accordance with some embodiments. In some embodiments, the method 1800 is performed at a computing device (e.g., computer system 130 or electronic device 160) that includes one or more processors (e.g., CPU(s) 302) and memory (e.g., memory 306). For example, in some embodiments, the computer device is a server or control console (e.g., a server, a standalone computer, a workstation, a smart phone, a tablet device, a medical system) that is in communication with a handheld ultrasound probe or ultrasound scanning system. In some embodiments, the computing device is a control unit integrated into a handheld ultrasound probe or ultrasound scanning system. Although the operations described in FIGS. 18A and 18B are described with respect to a computing device, in some embodiments, the operations described in method 1800 can be split among different devices (e.g., between a computer system 130 and an ultrasound device 200, between a computer system 130 and an electronic device 160, between an ultrasound device 200 and an electronic device 160, or between multiple computer systems 130). Additional details of the method 1800 can be found in FIGS. 1 to 17 and the accompanying descriptions, and are not repeated for the sake of brevity.

During a first scan performed by the ultrasound probe, the computing device receives (1802) a plurality of ultrasound images of a lung acquired via the ultrasound probe.

The computing device, while receiving the plurality of ultrasound images, for each ultrasound image of the plurality of ultrasound images, inputs (1804) a respective ultrasound image of the plurality of ultrasound images into a multi-headed model.

The computing device receives (1806), from a first head of the multi-headed model (e.g., first head 1330, FIG. 13) (e.g., segmentation portion), a first output comprising one or more segmented regions of the respective image.

The computing device receives (1808), from a second head of the multi-headed model (e.g., second head 1340, FIG. 13) (e.g., classification portion), a second output comprising a quality score associated with the respective image. The quality score is indicative of a quality of the respective image.

The computing device causes (1810) concurrent display of the quality score and the one or more segmented regions. This is illustrated in, for examples, FIGS. 15B to 15F.

In some embodiments, causing concurrent display of the quality score and the one or more segmented regions includes causing (1812) display of a gauge (e.g., gauge 1506), wherein the quality score is caused to be displayed as an output of the gauge (e.g., via indicator 1512).

In some embodiments, the computing device, in accordance with a determination the plurality of ultrasound images includes a threshold number of ultrasound images that each satisfies a threshold quality score, automatically (e.g., without user intervention) records (1814) a stack of first ultrasound images of the lung acquired via the ultrasound probe. See, as an example, Figured 15C to 15F.

With continued reference to FIG. 18B, in some embodiments, while automatically recording of the stack of first ultrasound images, for each first ultrasound image of the stack of first ultrasound images, the computing device, in accordance with a determination that the respective first ultrasound image satisfies the threshold quality score: (i) in accordance with a determination that a number of the first ultrasound images in the stack is less than a threshold number, continues (1816) the automatic recording of the stack; and (ii) in accordance with a determination that the number of the first ultrasound images in the stack satisfies the threshold number, ceases the awhile automatically recording of the stack.

In some embodiments, while automatically recording of the stack of first ultrasound images, for each first ultrasound image of the stack of first ultrasound images: the computing device, in accordance with a determination that the respective first ultrasound image does not satisfy the threshold quality score: (i) discards (1818) the respective image; and (ii) continues to record the stack of first ultrasound images.

In some embodiments, the computing device, while automatically recording the stack of first ultrasound images of the lung, obtains (1820) diagnostic information of the lung based on the stack of ultrasound images.

In some embodiments, the computing device, in accordance with a determination that the number of the first ultrasound images in the stack satisfies a threshold number: (i) ceases (1822) the automatic recording of the stack and (ii) causes display of a diagnosis of the lung. This is illustrated in, for example, FIGS. 15F and 15G.

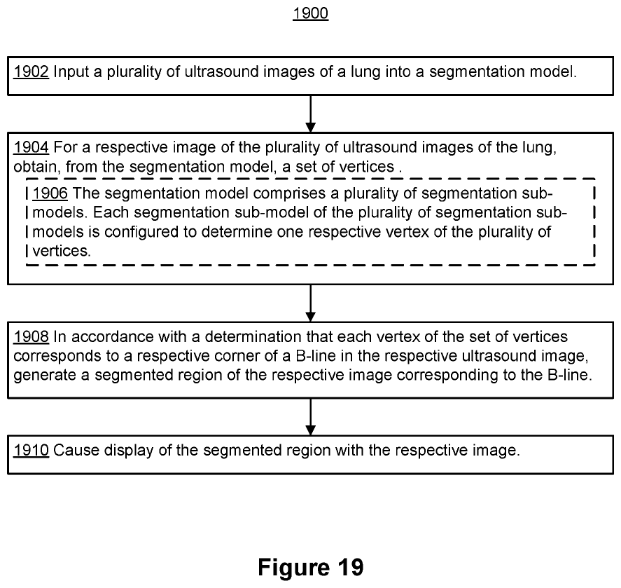

FIG. 19 illustrates a flowchart diagram for a method 1900 of detecting presence of B-lines in ultrasound images (e.g., lung ultrasound images), in accordance with some embodiments. In some embodiments, the method 1900 is performed at a computing device (e.g., computer system 130 or electronic device 160) that includes one or more processors (e.g., CPU(s) 302) and memory (e.g., memory 306). For example, in some embodiments, the computer device is a server or control console (e.g., a server, a standalone computer, a workstation, a smart phone, a tablet device, a medical system) that is in communication with a handheld ultrasound probe or ultrasound scanning system. In some embodiments, the computing device is a control unit integrated into a handheld ultrasound probe or ultrasound scanning system. Although the operations described in FIG. 19 are described with respect to a computing device, in some embodiments, the operations described in method 1900 can be split among different devices (e.g., between a computer system 130 and an ultrasound device 200, between a computer system 130 and an electronic device 160, between an ultrasound device 200 and an electronic device 160, or between multiple computer systems 130). Additional details of the method 1900 can be found in FIGS. 1 to 18B and the accompanying descriptions, and are not repeated for the sake of brevity.

The computing device inputs (1902) a plurality of ultrasound images (e.g., images 1402) of a lung into a segmentation model (e.g., B-line detection model 1410).

The computing device, for a respective image of the plurality of ultrasound images of the lung, obtains (1904) from the segmentation model a set of vertices (e.g., points) (e.g., vertex predictions 1414).

In some embodiments, the segmentation model comprises (1906) a plurality of segmentation sub-models (e.g., sub-models 1412). Each segmentation sub-model of the plurality of segmentation sub-models is configured to determine one respective vertex of the plurality of vertices.

The computing device, in accordance with a determination that each vertex of the set of vertices corresponds to a respective corner of a B-line in the respective ultrasound image, generates (1908) a segmented region of the respective image corresponding to the B-line.

The computing device causes (1910) display of the segmented region with the respective image.

Although some of various drawings illustrate a number of logical stages in a particular order, stages that are not order dependent may be reordered and other stages may be combined or broken out. While some reordering or other groupings are specifically mentioned, others will be obvious to those of ordinary skill in the art, so the ordering and groupings presented herein are not an exhaustive list of alternatives. Moreover, it should be recognized that the stages could be implemented in hardware, firmware, software or any combination thereof.

It will also be understood that, although the terms first, second, etc. are, in some instances, used herein to describe various elements, these elements should not be limited by these terms. These terms are only used to distinguish one element from another. For example, a first segmentation value could be termed a second segmentation value, and, similarly, a second segmentation value could be termed a first segmentation value, without departing from the scope of the various described implementations. The first segmentation value and the second segmentation value are both segmentation values, but they are not the same type of segmentation value.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

The foregoing description, for purpose of explanation, has been described with reference to specific implementations. However, the illustrative discussions above are not intended to be exhaustive or to limit the scope of the claims to the precise forms disclosed. Many modifications and variations are possible in view of the above teachings. The implementations were chosen in order to best explain the principles underlying the claims and their practical applications, to thereby enable others skilled in the art to best use the implementations with various modifications as are suited to the particular uses contemplated.

What is claimed is:

1. A method of detecting presence of A-lines in ultrasound images, comprising:
inputting a plurality of ultrasound images of a lung into a segmentation model;
for a respective image of the plurality of ultrasound images of the lung, obtaining, from a first output of the segmentation model, a first segmented region of the respective image corresponding to a candidate A-line;
comparing the first segmented region in the respective image corresponding to the candidate A-line to other segmented regions from other images in the plurality of ultrasound images to determine a second segmented region of the respective image corresponding to an actual A-line; and
causing display of the second segmented region of the respective image.

2. The method of claim 1, wherein:
the segmentation model is a multi-headed model, and
the multi-headed model includes:
a first head of the multi-headed model configured to output the first output; and
a second head of the multi-headed model configured to output a second output comprising a quality metric associated with the respective image.

3. The method of claim 1, wherein the first output comprises, for each respective portion of a plurality of portions of the image, a respective segmentation value of a plurality of segmentation values.

4. The method of claim 1, further comprising:
obtaining, from the segmentation model, a third segmented region of the respective image corresponding to a pleura of the lung;
using the third segmented region to generate a mask; and
using the mask to detect the presence or absence of B-lines in the respective image.

5. The method of claim 4, wherein causing display of the second segmented region of the respective image includes causing the third segmented region to be displayed concurrently with the second segmented region in the respective image.

6. The method of claim 4, further comprising:
obtaining, from the segmentation model, a fourth segmented region of the respective image corresponding to one or more ribs adjacent to the pleura of the lung.

7. The method of claim 6, wherein causing display of the second segmented region of the respective image includes causing the third segmented region and the fourth segmented region to be displayed concurrently with the second segmented region in the respective image.

8. The method of claim 1, wherein:
the respective image comprises a plurality of pixels; and
the segmentation model is configured to output, for a respective pixel of the plurality of pixels, a respective segmentation value corresponding to the respective pixel.

9. The method of claim 8, wherein:
the respective segmentation value is one of a plurality of segmentation values that the segmentation model is configured to output; and
the plurality of segmentation values includes a first segmentation value corresponding to a background, a second segmentation value corresponding to a pleura, and a third segmentation value corresponding to the A-line.

10. The method of claim 9, wherein the plurality of segmentation values includes a fourth segmentation value corresponding to one or more ribs adjacent to the pleura.

11. The method of claim 8, wherein
obtaining the first segmented region of the respective image corresponding to the candidate A-line comprises:
identifying, from the plurality of pixels, a first group of pixels that are contiguously located on the respective image, wherein each pixel of the first group of pixels has the same segmentation value corresponding to the A-line.

12. The method of claim 1, wherein
the respective image and the other images form a sequence of images in the plurality of ultrasound images.

13. The method of claim 1, further comprising:
prior to inputting the plurality of ultrasound images into the segmentation model, pre-processing the plurality of ultrasound images,
wherein the pre-processing includes at least one of: normalizing respective intensities of the plurality of ultrasound images and adjusting respective image sizes of the plurality of ultrasound images.

14. The method of claim 1, further comprising:
obtaining a first region corresponding to the respective image, the first region having a plurality of columns of pixels;
comparing respective intensity values from a respective column of pixels, of the plurality of columns of pixels, with other intensity values from other columns of pixels, of the plurality of columns of pixels in the first region; and
determining one or more candidate B-lines in the first region in accordance with the comparing.

15. The method of claim 14, further comprising:
applying a mask to the one or more candidate B-lines in the first region to obtain one or more actual B-lines in the first region.

16. The method of claim 15, further comprising:
causing the one or more actual B-lines to be displayed in the respective image.

17. The method of claim 16, wherein:
the respective image has a first coordinate system;
the first region is determined relative to a second coordinate system, distinct from the first coordinate system; and
causing display of the one or more actual B-lines in the respective image comprises transforming the one or more actual B-lines from the second coordinate system to the first coordinate system.

18. The method of claim 14, wherein:
the first region is obtained by transforming a first portion of the respective image, the first region having a rectilinear shape and the first portion having a conical shape.

19. (canceled)

20. A computer device, comprising:
one or more processors; and
memory storing instructions that, when executed by the one or more processors, cause the computer device to perform a set of operations, comprising:
inputting a plurality of ultrasound images of a lung into a segmentation model;
for a respective image of the plurality of ultrasound images of the lung, obtaining, from a first output of the segmentation model, a first segmented region of the respective image corresponding to a candidate A-line;
comparing the first segmented region in the respective image corresponding to the candidate A-line to other segmented regions from other images in the plurality of ultrasound images to determine a second segmented region of the respective image corresponding to an actual A-line; and
causing display of the second segmented region of the respective image.

21. A non-transitory computer-readable storage medium having stored thereon program code instructions that, when executed by a processor, cause the processor to perform a set of operations, comprising:
inputting a plurality of ultrasound images of a lung into a segmentation model;
for a respective image of the plurality of ultrasound images of the lung, obtaining, from a first output of the segmentation model, a first segmented region of the respective image corresponding to a candidate A-line;
comparing the first segmented region in the respective image corresponding to the candidate A-line to other segmented regions from other images in the plurality of ultrasound images to determine a second segmented region of the respective image corresponding to an actual A-line; and
causing display of the second segmented region of the respective image.

22.-32. (canceled)
